(19) United States
(12) Patent Application Publication
(10) Pub. No.: US 2018/0071376 A1
(43) Pub. Date: Mar. 15, 2018

(54) TOLEROGENIC NANOPARTICLES FOR TREATING DIABETES MELLITUS

(71) Applicant: The Brigham and Women`s Hospital, Inc., Boston, MA (US)

(72) Inventors: Ada Yeste Bornal, 08030 (ES); Francisco J. Quintana, Jamaica Plain, MA (US)

(21) Appl. No.: 15/560,276

(22) PCT Filed: Mar. 23, 2016
(86) PCT No.: PCT/US16/23845

§ 371 Date: Sep. 21, 2017

Related U.S. Application Data
(60) Provisional application No. 62/136,897, filed on Mar. 23, 2015.

(51) Int. Cl. A61K 39/00; A61K 31/427; A61K 45/06; A61K 35/17; A61K 31/135; A61K 47/69; A61K 47/52

(52) U.S. Cl. CPC A61K 39/0008; A61K 31/427; A61K 45/06; A61K 2039/577; A61K 31/135; A61K 47/6929; A61K 47/52; A61K 35/17

(57) ABSTRACT

Methods and compositions for increasing the number and/or activity of regulatory T cells (Tregs) in vivo and in vitro, to induce tolerance to diabetogenic autoantigens.

### CLAIM OF PRIORITY

This application claims the benefit of U.S. Patent Application Ser. No. 62/136,897, filed on Mar. 23, 2015. The entire contents of the foregoing are hereby incorporated by reference.

### FEDERALLY SPONSORED RESEARCH OR DEVELOPMENT

This invention was made with Government support under Grant No. AI093903 awarded by the National Institutes of Health. The Government has certain rights in the invention.

### TECHNICAL FIELD

This invention relates to methods and compositions for increasing the number and/or activity of regulatory T cells (Tregs) in vivo and in vitro, to induce tolerance to diabetogenic autoantigens.

### BACKGROUND

Type 1 diabetes (T1D) is a T-cell mediated autoimmune disease characterized by the destruction of insulin-producing β cells in the pancreas¹⁻⁶. Therefore, the reestablishment of immune tolerance is a major goal for the treatment of T1D⁷. Immunologic tolerance is mediated by a number of mechanisms including deletion, anergy, and active regulation by specialized regulatory cells. Several regulatory T-cell (Treg) subsets mediate immune tolerance, of particular importance are FoxP3+ Tregs^{8, 9}. Indeed, deficits in pancreatic Tregs, both in numbers and functionality, have been described in recent onset T1D subjects^{10, 11}. In addition, it has been reported that effector T cells in T1D subjects are resistant to regulation by Tregs, suggesting that specific Treg subpopulations are required to control diabetogenic T cells¹²⁻¹⁴. Conversely, therapies that increase Treg numbers and function prevent and treat T1D in pre-clinical models, and are currently under investigation in clinical trials¹²⁻¹⁴.

The administration of ex vivo expanded Tregs or tolerogenic dendritic cells (DCs) that promote Treg expansion in vivo is a potential therapeutic approach for T1D, but cell-based therapeutic approaches are hard to translate into clinical practice¹⁵⁻²⁰. Alternatively, antibody- and cytokine-based therapies have been developed to restore immune-tolerance and suppress autoimmunity in T1D^{5, 20-23}. However, these approaches can lead to generalized immune suppression. Thus, there is an unmet clinical need for approaches to induce functional antigen-specific Tregs in vivo as method for the re-establishment of immune tolerance in T1D and other autoimmune disorders.

### SUMMARY

Regulatory T cells (Tregs) are involved in the suppression of immune responses and autoimmunity. Deficits in Tregs have been reported in type 1 diabetes (T1D); thus, the induction of functional Tregs represents a potential approach to reestablish tolerance in those settings. Aryl hydrocarbon receptor (AhR) is a transcription factor that upon activation by its ligand 2-(1′1-indole-3′-carbonyl)-thiazole-4-carboxylic acid methyl ester (ITE) or other ligands induces tolerogenic dendritic cells (DCs) that promote the generation of regulatory T cells. The present invention is based, at least in part, on the discovery that nanoparticles (NPs) can be used to co-deliver a small tolerogenic molecule (ITE) and the pancreatic antigen Insulin (NP_{ITE+Ins}) and induce the generation of Tregs by DCs both in vitro and in vivo. NP_{ITE+Ins} suppressed the spontaneous development of T1D in non-obese diabetic (NOD) mice. Thus, described herein are NPs and methods for use thereof to reestablish tolerance for the prevention or treatment of Type 1 diabetes caused by an abnormal (autoimmune) response to pancreatic antigens.

Thus, provided herein are compositions comprising: (i) a ligand that binds specifically to an aryl hydrocarbon receptor (AHR) transcription factor, and (ii) a diabetes autoantigen, wherein both (i) and (ii) are linked to a biocompatible nanoparticle.

In some embodiments, the ligand is a small molecule ligand of AHR, e.g., a ligand described herein, e.g., 2,3,7,8 tetrachlorodibenzo-p-dioxin (TCDD), tryptamine (TA), laquinimod, 6 formylindolo[3,2 b]carbazole (FICZ), and/or 2-(1′H-indole-3′-carbonyl)-thiazole-4-carboxylic acid methyl ester (ITE).

In some embodiments, the ligand is ITE.

In some embodiments, the diabetes autoantigen is selected from the group consisting of preproinsulin or an immunologically active fragment thereof, islet cell autoantigens (ICA), glutamic acid decarboxylase (GAD), islet tyrosine phosphatase ICA512/IA-2, ICA12, ICA69, HSP60, HSP70, IGRP, carboxypeptidase H, peripherin, and gangliosides, or immunologically active fragments thereof.

In some embodiments, the diabetes autoantigen is selected from the group consisting of preproinsulin or an immunologically active fragment thereof, islet cell autoantigen (ICA), or GAD.

In some embodiments, the composition includes a monoamine oxidase inhibitor (MAOI). In some embodiments, the MAOI is a hydrazine such as isocarboxazid; nialamide; phenelzine; or hydracarbazine; or tranylcypromine.

In some embodiments, the composition includes an antibody that selectively binds to an antigen present on a T cell, a B cell, a dendritic cell, or a macrophage. In some embodiments, the antibody is selected from the group consisting of antibodies that bind specifically to CXCR4, CD28, CD8, CTLA4, CD3, CD20, CD19, CD11c, DEC205, MHC class I or class II, CD80, CD86, CD11b, MHC class I or class II, CD80, or CD86. In some embodiments, the antibody is linked to the biocompatible nanoparticle.

Also provided herein are methods for increasing the number of CD4/CD25/Foxp3-expressing T regulatory (Treg) cells in a population of T cells. The methods include contacting the population of cells with a sufficient amount of the composition of claim 1, and optionally evaluating the presence and/or number of CD4/CD25/Foxp3-expressing cells in the population; wherein the method results in an increase in the number and/or activity of regulatory T cells (Treg).

In some embodiments, the population of T cells comprises naïve T cells or CD4⁺CD62 ligand⁺ T cells.

In some embodiments, the methods include administering the Treg cells to a subject suffering from or at risk of developing diabetes.

Also provided herein are methods, and the use of the compositions described herein, for treating, preventing, or reducing the risk of developing type 1 diabetes in a subject. The methods include administering to the subject a therapeutically effective amount of a composition described herein. In some embodiments, the methods include administering to the subject a therapeutically effective amount of a composition described herein, plus one or both of a monoamine oxidase inhibitor (MAOI), and an antibody that selectively binds to an antigen present on a T cell, a B cell, a dendritic cell, or a macrophage.

In some embodiments, the antibody is selected from the group consisting of antibodies that bind specifically to CXCR4, CD28, CD8, CTLA4, CD3, CD20, CD19, CD11c, DEC205, MHC class I or class II, CD80, CD86, CD11b, MHC class I or class II, CD80, or CD86.

In some embodiments, the MAOI is a hydrazine such as isocarboxazid; nialamide; phenelzine; or hydracarbazine; or tranylcypromine.

In some embodiments, levels of IL-10 producing T cells (Tr1 cells) and/or IL-10 producing CD8 T cells are increased in the subject.

As used herein, “treatment” means any manner in which one or more of the symptoms of a disease or disorder are ameliorated or otherwise beneficially altered. As used herein, amelioration of the symptoms of a particular disorder refers to any lessening of the symptoms, whether permanent or temporary, lasting or transient, that can be attributed to or associated with treatment by the compositions and methods of the present invention.

The terms “effective amount” and “effective to treat,” as used herein, refer to an amount or a concentration of one or more of the compositions described herein utilized for a period of time (including acute or chronic administration and periodic or continuous administration) that is effective within the context of its administration for causing an intended effect or physiological outcome.

The term “subject” is used throughout the specification to describe an animal, human or non-human, rodent or non-rodent, to whom treatment according to the methods of the present invention is provided. Veterinary and non-veterinary applications are contemplated. The term includes, but is not limited to, mammals, e.g., humans, other primates, pigs, rodents such as mice and rats, rabbits, guinea pigs, hamsters, cows, horses, cats, dogs, sheep and goats. Typical subjects include humans, farm animals, and domestic pets such as cats and dogs.

The term gene, as used herein refers to an isolated or purified gene. The terms “isolated” or “purified,” when applied to a nucleic acid molecule or gene, includes nucleic acid molecules that are separated from other materials, including other nucleic acids, which are present in the natural source of the nucleic acid molecule. An “isolated” nucleic acid molecule, such as an mRNA or cDNA molecule, can be substantially free of other cellular material, or culture medium when produced by recombinant techniques, or substantially free of chemical precursors or other chemicals when chemically synthesized.

An “isolated” or “purified” polypeptide, peptide, or protein is substantially free of cellular material or other contaminating proteins from the cell or tissue source from which the protein is derived, or substantially free from chemical precursors or other chemicals when chemically synthesized. “Substantially free” means that the preparation of a selected protein has less than about 30%, (e.g., less than 20%, 10%, or 5%) by dry weight, of non-selected protein or of chemical precursors. Such a non-selected protein is also referred to herein as “contaminating protein”. When the isolated therapeutic proteins, peptides, or polypeptides are recombinantly produced, it can be substantially free of culture medium, i.e., culture medium represents less than about 20%, (e.g., less than about 10% or 5%) of the volume of the protein preparation. The invention includes isolated or purified preparations of at least 0.01, 0.1, 1.0, and 10 milligrams in dry weight.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-I. NP_{ITE+MIMO} induces tolerogenic DCs. (A) Transmission EM analysis of uptake of NP_{ITE+MIMO} by splenic DCs in culture. (B) Analysis of cyp1a1 expression by DCs coincubated with NPs 24 h after initiation of cell cultures. (C) FACS analysis of DCs incubated in vitro with NPs and activated with LPS for 24 h. (D) Quantitative PCR analysis of il6 and il2 expression in DCs incubated in vitro with NPs and activated with LPS for 24 h; results presented relative to gapdh mRNA. (E-H) DCs were coincubated in vitro with NPs, activated with LPS, and used to stimulate naive BDC2.5+ CD4+ T cells. Proliferation (E) and cytokine secretion (F) in the supernatants were analyzed at 72 and 48 h, respectively. (G) Quantitative PCR analysis of ifng and il17 was also performed at 12h. (H) The frequency of CD4+ FoxP3+, IFNγ+ or IL-17+ CD4+ cells was analyzed by FACS at 72 h. (I) Ratios between FoxP3+ and IFNγ+ or IL-17+ T cells. Representative data of one of three experiments that produced similar results (*P<0.05, **P<0.01, and ***P<0.001).

FIGS. 2A-H. NP_{ITE+Ins} suppress T1D in NOD mice. Eight week old NOD mice were treated with NP NP_{ITE+Ins} once a week (A) Diabetes incidence (%) and (B) glycaemia values (mg/dl) in NOD mice treated i.p weekly for 1 month with NP_{ITE+Ins}. (C) Histology of pancreas. (D) Quantitative PCR analysis of rorc, tbet, foxp3, ifng and i117 of pancreatic lymphnodes. (E) Serum IgG and IgM autoantibody signature in serum. (F-H) BDC2.5 NOD mice were treated weekly for 1 month with NP or NP_{ITE+MIMO}. (F) Frequency of IFNγ+ and IL-17+ CD4+ T cells. (G) Quantitative PCR of foxp3 and (H) FACS analysis of FoxP3+ CD4+ T cells. (*P<0.05, **P<0.01, and ***P<0.001).

FIGS. 3A-B. NP_{ITE+MIMO} induces tolerogenic DCs in vivo. NOD mice were treated once a week for 1 month with NP or NP_{ITE+MIMO}. (A) Quantitative PCR analysis of cyp1a1 from splenic DCs. (B) Quantitative PCR analysis of i16 and i112 on splenic DC activated with LPS for 6h. (*P<0.05 and **P<0.01).

FIG. 4A-G. AhR activation by NP_{ITE+Ins} controls socs2 expression and DC activation. (A) Gene expression analysis was performed in splenic DCs from NP or NP_{ITE+Ins}-treated NOD mice (in vivo, left panel) and BMDCs treated with NP or NP_{ITE+Ins} (in vitro, right panel). (B) Signaling pathways targeted by NP_{ITE+Ins} in DCs. (C) Quantitative PCR analysis of socs2 in splenic DCs treated with NP or NP_{ITE+Ins}. (D) Immunoblot analysis of TRAF6, (E) AhR responsive elements (XRE-1,2,3) in the socs2 promoter (Left). Chromatin-immunoprecipitation analysis of the interaction of AhR with XRE-1,2,3 in the socs2 promoter. (F) The upregulation of socs2 expression in DCs triggered by NP_{ITE+Ins} is mediated by AhR. Socs2 expression in DCs from WT or AhR hypomorphic (AhR-d) mice treated in vitro with NP or NP_{ITE+Ins}. (*P<0.05, **P<0.01, and ***P<0.001). (G) Effect of NP_{ITE+Ins} on the activation of NF-kb and Erk1/2 in DCs.

FIGS. 5A-C. Socs2 expression in DCs mediates the regulation of DC function by NP_{ITE+Ins}. Socs2 was knocked down in BMDCs and then cells were incubated with NP or NP_{ITE+Ins}. (A) Immunoblot analysis and (B) quantification of p65 in BMDCs incubated with LPS for 1 h. (C) FACS analysis of IL-17+ and IFNγ+ CD4+ T cells activated with NP_{ITE+Ins}-treated BMDC or BMDC-KD. (*P<0.05, **P<0.01, and ***P<0.001).

FIGS. 6A-C. NP_{ITE+GAD} induce tolerogenic DCs in humans. Immature and mature human monocyte-derived dendritic cells (hDCs) were incubated with NP, NP_{ITE}, NP_{GAD}, NP_{ITE+GAD} for 24h. (A) Gene expression and (B) FACS analysis of CD40, CD80, CD86 and HLA-DR in immature hDCs treated with NP, NP_{ITE}, NP_{GAD}, NP_{ITE+GAD}. These cells were then used to stimulate human CD4+ T cells. (C) IFNγ and IL-17 production in the supernatant by the CD4+ T cells was quantified by ELISA.

FIGS. 7A-D. Characterization of NPs containing ITE and Insulin or MIMO. (A) Schematic representation of NP_{ITE+Ins}. (B) Transmission EM analysis of pegylated NPs. (C) HEK293 cells transfected with a reporter construct coding for luciferase under the control of an AhR-responsive promoter were incubated with NPs and luciferase activity was measured after 24 h. Cotransfection with a TK-Renilla construct was used for normalization purposes. (D) Quantification if Insulin and MIMO incorporation in the NPs. (*P<0.05, **P<0.01, and ***P<0.001).

### DETAILED DESCRIPTION

The ligand-activated transcription factor aryl hydrocarbon receptor (AhR) controls the development of effector and Tregs²⁴⁻³¹. Administration of the non-toxic AhR ligand 2-(1′H-indole-3′-carbonyl)-thiazole-4-carboxylic acid methyl ester (ITE)³² induces functional Tregs that suppress experimental autoimmunity²⁸. Indeed, AhR activation induces a tolerogenic phenotype in DCs that promotes the differentiation of Tregs^{28, 30, 33-36}. Nanoparticles (NPs) engineered to co-deliver a tissue-specific antigen and ITE to DCs in vivo re-established antigen-specific tolerance in the experimental autoimmune encephalomyelitis model (EAE) of multiple sclerosis³⁰ and WO 2009/067349. However, that model is one in which the antigen is clearly defined, because of the nature of the model. To determine whether these methods could be used for the reestablishment of immune tolerance in T1D, where the autoantigen can vary, the effects of NPs on T1D were studied in the non-obese mouse (NOD) model. As shown herein, NPs loaded with the tolerogenic AhR ligand ITE and the β-cell antigen insulin induce tolerogenic DCs in vivo that expanded the Treg compartment and arrest the development of NOD T1D. These data demonstrate the efficacy of NPs in arresting spontaneous autoimmune diabetes characterized by asynchronous initiation and onset. Thus. NPs loaded with autoantigens and tolerogenic molecules offer a new therapeutic tool to reestablish immune tolerance in autoimmune disorders.

The re-establishment of antigen specific tolerance is considered a potential therapeutic approach for T1D. We found that nanoparticle based co-administration of a β-cell antigen and the tolerogenic AHR ligand ITE suppressed the development of spontaneous NOD T1D. These protective effects involved the control of the diabetogenic immune response by tolerogenic DCs and regulatory T cells. Considering that in these studies treatment was initiated at the age of 8-10 weeks at which insulitis is already detectable⁵¹, our results suggest that NPs provide a therapeutic avenue to re-establish antigen specific tolerance in AID and other immune-mediated diseases.

Th1 and Th17 cells can both induce diabetes in NOD recipients⁵². However, the induction of diabetes by Th17 cells is associated with the co-expression of IFNγ⁵². Moreover, Tbet expression in T cells is required for T1D development in NOD mice⁵³ and IL-17 knock down ameliorates CNS autoimmunity but fails to affect T1D development in NOD mice⁵⁴. Taken together, these data suggest that Th1 cells play a dominant role in the NOD diabetogenic response. Interestingly, the arrest of diabetes with NP_{ITE+Ins} was linked to a stronger suppression of the Th1 response. Since in vitro NP_{ITE+Ins} inhibited both Th1 and Th17 polarizing cytokines, these observations likely reflect increased susceptibility of Th1 cells to inhibition in vivo and/or the differential targeting of specific Th1-inducing APCs. In addition, considering that NOD T1D is also driven by CD8+ T cells⁶, our data suggest that NP_{ITE+Ins} also control the CD8+ T cell response.

Several types of regulatory T cells have been shown to control T1D development. Among these different types, FoxP3+ Tregs play a significant role. Treg deficits have been associated to T1D in mice and humans⁵⁵, and FoxP3+ Treg removal results in T1D acceleration in NOD mice⁵⁶. Conversely, FoxP3+ Treg transfer or induction in vivo interfere with NOD T1D development^{57, 58}. The present data indicates that NP expand FoxP3+ Tregs and transfer protection from NOD T1D. It is possible, however, that the protective effects of NPs also involve other regulatory T cell populations such as IL-10+ CD4+ Tr1 cells and CD8+ Tregs. Indeed, antigen-specific anti-diabetogenic CD8+ Tregs induced by NPs have been shown to arrest NOD T1D⁵⁹. However, these CD8+ Tregs are induced in a FoxP3+ Treg-dependent manner^{60, 61}. The induction of several T cell population with regulatory activity is likely to result in an increased ability to control a pre-existing diabetogenic immune response, as it has been shown that different Tregs subpopulations can control different stages and aspects of the autoimmune response^{8, 62, 63}.

Without wishing to be bound by theory, it is believed that the protective effects of NPs on NOD T1D involved tolerogenic DCs. Several types of tolerogenic DCs have been shown to control inflammation⁶⁴. Moreover, several pathways have been shown to control anti-inflammatory functions in DCs. For example, IL-27 signaling in DCs has been shown to limit T cell mediated inflammation⁴⁵. The tolerogenic effects of NPs in this work were mediated by the activation of AHR, and are indeed in agreement with previous reports of anti-inflammatory effects of Ahr signaling in DCs^{28, 30, 35, 36, 65}. Although those previous investigations linked IDO and RA to the anti-inflammatory effects of AHR signaling in DCs, the present work identifies SOCS2 as an additional pathway exploited by AHR to mediate its tolerogenic effects in DCs through the inhibition of TRAF6-mediated NF-kB activation and potentially, its degradation by the immunoproteasome. Socs2 signaling in T cells has been previously linked to the induction of Foxp3+ Tregs in response to anti-CD3 treatment⁶⁶, but the relationship between SoCS2 in DCs and Tregs is previously unknown. This observation suggests that the effects of Ahr in DCs might be broader than anticipated and might involve additional disease and/or tissue specific mechanisms. Moreover, although AHR signaling clearly diminished p38 and Erk1 activation, these effects were independent of SOCS2, suggesting that additional anti-infalmmatory pathways might be triggered by AhR in DCs. As we already mentioned, NPs affected NF-kB signaling in an AHR dependent manner. The regulation of NF-kB activity has been linked to T1D immunopathology^{67, 68}. Thus, improved NPs could potentially be engineered to target additional pathways known to regulate NF-kb activity, such as p38⁶⁹, and therefore tune their immunomodulatory.

Several strategies have been attempted to modulate antigen specific responses in vivo, many of them in the course of T1D. Tolerogenic antigen administration has been found to arrest antigen-specific T cell responses in mice and t1D subjects, but its success in the arrest of T1D has been limited⁷⁰. The tolerogenic potential of these experimental therapies has been boosted with the co-administration of antigen fused to DC-targeting antigens⁷⁰. Antigen administration with coding DNA vaccines has also been shown to work on experimental models of autoimmunity, and has shown promising results in human trials⁷¹⁻⁷³. More recently, nanoparticle-administered antigens have been shown to induce tolerance^{30, 59, 74}. The strategy described in this manuscript seeks to further enhance tolerance by co-administering the antigen with a tolerogenic molecule targeting AhR using a nanoparticle-based approach. This modular system allows the incorporation of additional tolerogenic molecules to further enforce tolerance or induce specific Treg populations, as well as the sue of targeting antibodies to target the NPs to specific cell types⁷⁵, therefore providing ample room for its optimization to increase its translational value. In combination with methods for the monitoring of the immune response of individual T1D subjects^{47, 76-78} to chose the relevant antigens for each subject and monitor the effect of tolerization, NPs offer a new therapeutic avenue for T1D and other autoimmune diseases.

The data presented herein demonstrates the use of nanoparticles that co-deliver diabetes autoantigens and AHR-specific ligands, e.g., the high affinity AHR ligand 2,3,7,8-tetrachlorodibenzo-p-dioxin (TCDD), tryptamine (TA), and/or 2-(1′H-indole-3′-carbonyl)-thiazole-4-carboxylic acid methyl ester (ITE), to promote an increase in the number and/or activity of Treg immunomodulatory cells, which will be useful to suppress the autoimmune response to treatment, prevent, or reduce the risk of Type 1 diabetes.

Other potentially useful AHR transcription factor ligands are described in Denison and Nagy, Ann. Rev. Pharmacol. Toxicol., 43:309-34, 2003, and references cited herein, all of which are incorporated herein in their entirety. Other such molecules include planar, hydrophobic HAHs (such as the polyhalogenated dibenzo-pdioxins, dibenzofurans, and biphenyls) and PAHs (such as 3-methylcholanthrene, benzo(a)pyrene, benzanthracenes, and benzoflavones), and related compounds. (Denison and Nagy, 2003, supra). Nagy et al., Toxicol. Sci. 65:200-10 (2002), described a high-throughput screen useful for identifying and confirming other ligands. See also Nagy et al., Biochem. 41:861-68 (2002). In some embodiments, those ligands useful in the present invention are those that bind competitively with TCDD, TA, and/or ITE.

### AHR Ligand-Nanoparticles

As demonstrated herein, compositions comprising nanoparticles linked to AHR ligands and diabetes autoantigens are surprisingly effective in delivering the ligand, both orally and by injection, and in inducing the Treg response in living animals. Thus, the invention further includes compositions comprising AHR ligands and diabetes autoantigens linked to biocompatible nanoparticles, optionally with antibodies that target the nanoparticles to selected cells or tissues.

AHR Transcription Factor Ligands

AHR-specific ligands, e.g., the high affinity AHR ligand 2,3,7,8-tetrachloro-dibenzo-p-dioxin (TCDD), tryptamine (TA), 6 formylindolo[3,2 b]carbazole (FICZ), and/or 2-(1′H-indole-3′-carbonyl)-thiazole-4-carboxylic acid methyl ester (ITE), promote an increase in the number and/or activity of Treg immunomodulatory cells, which will be useful to suppress the immune response in the treatment of diseases or disorders caused by an abnormal (e.g., an excessive, elevated, or inappropriate) immune response, e.g., an autoimmune disease or disorder. A number of small molecule AHR ligands are known in the art, including the following.

AHR ligands can also include structural analogs of 2-(1′H-indole-3′-carbonyl)-thiazole-4-carboxylic acid methyl ester (ITE), e.g., having following formula:

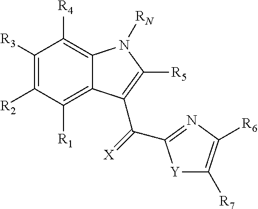

wherein X and Y, independently, can be either O (oxygen) or S (sulfur); RN can be selected from hydrogen, halo, cyano, formyl, alkyl, haloalkyl, alkenyl, alkynyl, alkanoyl, haloalkanoyl, or a nitrogen protective group; R₁, R₂, R₃, R₄, and R₅ can be independently selected from hydrogen, halo, hydroxy (—OH), thiol (—SH), cyano (—CN), formyl (—CHO), alkyl, haloalkyl, alkenyl, alkynyl, amino, nitro (—NO₂), alkoxy, haloalkoxy, thioalkoxy, alkanoyl, haloalkanoyl, or carbonyloxy; R₆ and R₇, can be independently selected from hydrogen, halo, hydroxy, thiol, cyano, formyl, alkyl, haloalkyl, alkenyl, alkynyl, amino, nitro, alkoxy, haloalkoxy, or thioalkoxy; or R₆ and R₇, independently, can be:

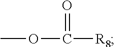

wherein R₅ can be selected from hydrogen, halo, cyano, alkyl, haloalkyl, alkenyl, or alkynyl; or R₆ and R₇, independently, can be:

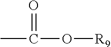

wherein R₉ can be selected from hydrogen, halo, alkyl, haloalkyl, alkenyl, or alkynyl; or R₆ and R₇, independently, can be:

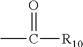

wherein R₁₀ can be selected from hydrogen, halo, hydroxy, thiol, cyano, alkyl, haloalkyl, alkenyl, alkynyl, amino, or nitro; or R₆ and R₇, independently, can also be:

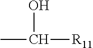

wherein R₁₁ can be selected from hydrogen, halo, alkyl, haloalkyl, alkenyl, or alkynyl.

In some embodiments, the structure of the 2-(1′H-indole-3′-carbonyl)-thiazole-4-carboxylic acid methyl ester analog is represented by the following structural formula:

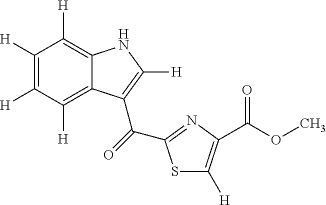 [SMILES: COC(=O)c1csc(C(=O)c2c[nH]c3ccccc23)n1]

In some further embodiments, the structural analog of 2-(1′H-indole-3′-carbonyl)-thiazole-4-carboxylic acid methyl ester is represented by the following structural formula:

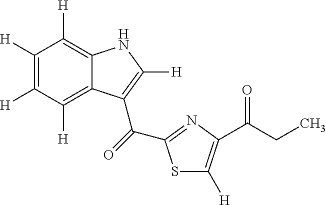 [SMILES: CCC(=O)c1csc(C(=O)c2c[nH]c3ccccc23)n1]

In some embodiments, the structural analog of 2-(1′H-indole-3′-carbonyl)-thiazole-4-carboxylic acid methyl ester is represented by the following structural formula:

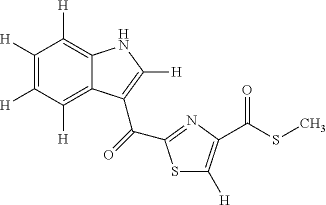 [SMILES: CSC(=O)c1csc(C(=O)c2c[nH]c3ccccc23)n1]

### See US20130338201 and US20130310429.

Other potentially useful AHR transcription factor ligands are described in Denison and Nagy, Ann. Rev. Pharmacol. Toxicol., 43:309-34, 2003, and references cited herein, all of which are incorporated herein in their entirety. Other such molecules include polycyclic aromatic hydrocarbons exemplified by 3-methylchoranthrene (3-MC); halogenated aromatic hydrocarbons typified by 2,3,7,8-tetrachlorodibenzo-p-dioxin (TCDD); planar, hydrophobic HAHs (such as the polyhalogenated dibenzo-pdioxins, dibenzofurans (e.g., 6-methyl-1,3,8-trichlorodibenzofuran (6-MCDF), 8-methyl-1,3,6-trichlorodibenzofuran (8-MCDF)), and biphenyls) and PAHs (such as 3-methylcholanthrene, benzo(a)pyrene, benzanthracenes, and benzoflavones), and related compounds. (Denison and Nagy, 2003, supra).

Naturally-occurring AHR ligands can also be used, e.g., tryptophan catabolites such as indole-3-acetaldehyde (IAAlD), indole-3-aldehyde (IAlD), indole-3-acetic acid (IAA), tryptamine (TrA), kynurenine, kynurenic acid, xanthurenic acid, 5-hydroxytryptophan, serotonin; and Cinnabarinic Acid (Lowe et al., PLoS ONE 9(2): e87877; Zelante et al., Immunity 39, 372-385, Aug. 22, 2013; Nguyen et al., Front Immunol. 2014 Oct. 29; 5:551); biliverdin or bilirubin (Quintana and Sherr, Pharmacol Rev 65:1148-1161, October 2013); prostaglandins (PGF3a, PGG2, PGH1, PGB3, PGD3, and PGH2); leukotrienes, (6-trans-LTB 4, 6-trans-12-epi-LTB); dihydroxyeicosatriaenoic acids (4,5(S),6(S)-DiHETE, 5(S),6(R)-DiHETE); hydroxyeicosatrienoic acid (12(R)-HETE) and lipoxin A4 (Quintana and Sherr, Pharmacol Rev 65:1148-1161, October 2013).

In some embodiments, the AHR ligand is a flavone or derivative thereof, e.g., 3,4-dimethoxyflavone, 3′-methoxy-4′-nitroflavone, 4′,5,7-Trihydroxyflavone (apigenin) or 1-Methyl-N-[2-methyl-4-[2-(2-methylphenyl)diazenyl]phenyl-1H-pyrazole-5-carboxamide; resveratrol (trans-3,5,4′-Trihydroxystilbene) or a derivative thereof; epigallocatechin or epigallocatechingallate.

In some embodiments, the AHR ligand is one of the 1, 2-dihydro-4-hydroxy-2-oxo-quinoline-3-carboxanilides, their thieno-pyridone analogs, and prodrugs thereof, e.g., having the structure

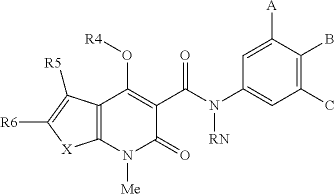

wherein
A, B and C are independently chosen from the group comprising H, Me, Et, iso-Pr, tert-Bu, OMe, OEt, O-iso-Pr, SMe, S(0)Me, S(0)₂ e, CF₃, 0CF₃, F, CI, Br, I, and CN, or A and B represents OCH₂0 and C is H;
RN is chosen from the group comprising H, C(0)H, C(0)Me, C(0)Et, C(0)Pr, C(0)CH(Me)₂, C(0)C(Me)₃, C(0)Ph, C(0)CH₂Ph, Cb0₂H, Cb0₂Me, Cb0₂Et, Cb0₂CH₂Ph, C(0)NHMe, C(0)NMe₂, C(0)NHEt, C(0)NEt₂, C(0)NHPh, C(O)NHCH₂Ph, the acyl residues of C5-C20 carboxylic acids optionally containing 1-3 multiple bonds, and the acyl residues of the amino acids glycine, alanine, valine, leucine, iso-leucine, serine, threonine, cysteine, methionine, proline, asparagine, glutamine, aspartic acid, glutamic acid, lysine, arginine, histidine, phenylalanine, tyrosine, and tryptophan, and optionally substituted 1-3 times by substituents chosen from the group comprising Me, Et, OMe, OEt, SMe, S(0)Me, S(0)₂Me, S(0)₂NMe₂, CF₃, OCF₃, F, CI, OH, C0₂H, C0₂Me, C0₂Et, C(0)NH₂, C(0)NMe₂, NH₂, NH₃\NMe₂, NMe₃⁺, NHC(O)Me, NC(═NH)NH₂, OS(0)₂₀H, S(0)₂0H, OP(0)(OH)₂, and P(0) (0H)₂;
R4 is RN, or when RN is H, then R₄ is chosen from the group comprising H, P(0) (OH)₂, P(O) (0Me)₂, P(0) (OEt)₂, P(O) (OPh)₂, P(0) (OCH₂Ph)₂, S(0)₂0H, S(0)₂NH₂, S(0)₂NMe₂, C(0)H, C(0)Me, C(0)Et, C(0)Pr, C(0)CH(Me)₂, C(0)C(Me)₃, C(0)Ph, C(0)CH₂Ph, C0₂H, C0₂Me, C0₂Et, C0₂CH₂Ph, C(0)NHMe, C(0)NMe₂, C(0)NHEt, C(0)NEt₂, C(0)NHPh, C(O)NHCH₂Ph, the acyl residues of C5-C20 carboxylic acids optionally containing 1-3 multiple bonds, and the acyl residues of the amino acids glycine, alanine, valine, leucine, iso-leucine, serine, threonine, cysteine, methionine, proline, asparagine, glutamine, aspartic acid, glutamic acid, lysine, arginine, histidine, phenylalanine, tyrosine, and tryptophan, and optionally substituted 1-3 times by substituents chosen from the group comprising Me, Et, OMe, OEt, SMe, S(0)Me, S(0)₂Me, S(0)₂NMe₂, CF₃, 0CF₃, F, CI, OH, C0₂H, C0₂Me, C0₂Et, C(0)NH₂, C(0) Me₂, NH₂, NH₃⁺, Me₂, NMe₃⁺, NHC(0)Me, NC(═NH)NH₂, OS(0)₂OH, S(0)₂OH, OP (0) (OH)₂, and P (0) (OH)₂; R5 and R6 are independently chosen from the group comprising H, Me, Et, iso-Pr, tert-Bu, OMe, OEt, 0-iso-Pr, SMe, S(0)Me, S(0)₂Me, CF₃, OCF₃, F, Cl, Br, I, and CN, or R5 and R6 represents OCH₂0; and X is —CH═CH—, or S, or pharmaceutically acceptable salts of the compounds thereof. See WO2012050500. In some embodiments, the AHR ligand is laquinimod (a 5-Cl, N-Et carboxanilide derivative) or a salt thereof (see, e.g., US20140128430).

In some embodiments, the AHR ligand is a small molecule characterized by the following general formula:

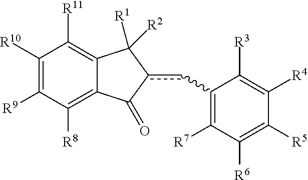

wherein (i) R₁ and R₂ independently of each other are hydrogen or a C₁ to C12 alkyl, (ii) R₃ to R₁₁ independently from each other are hydrogen, a C₁ to C₁₂ alkyl, hydroxyl or a C₁ to C₁₂ alkoxy, and (iii) the broken line represents either a double bond or two hydrogens. In some embodiments, the ligand has one of the following formulae:

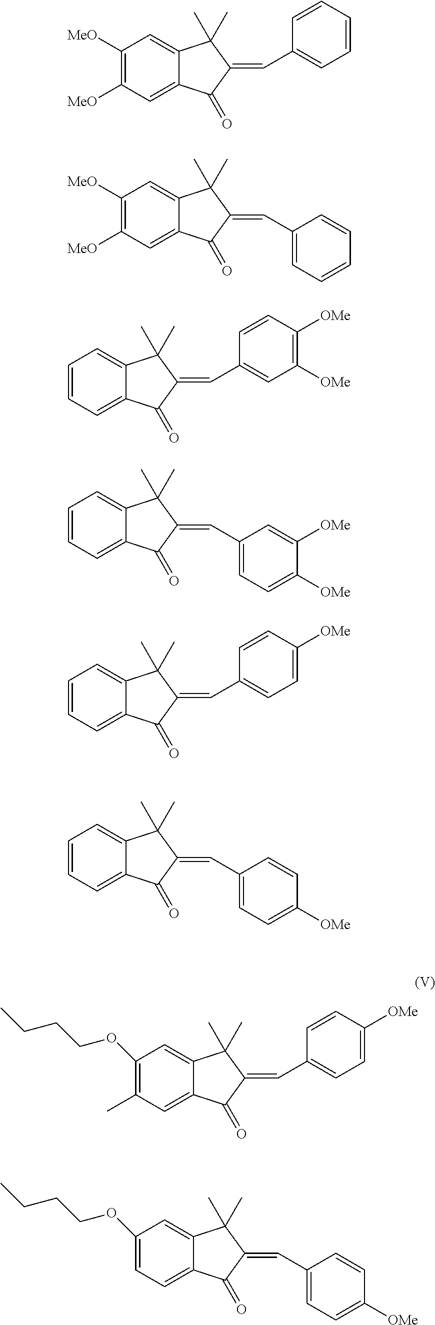

### See WO2007/128723 and US20140294860.

In some embodiments, the AhR ligand has a formula of:

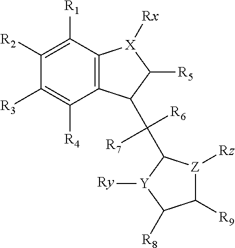

wherein:
R₁, R₂, R₃ and R₄ can be independently selected from the group consisting of hydrogen, halo, hydroxy (—OH), thiol (—SH), cyano (—CN), formyl (—CHO), alkyl, haloalkyl, alkenyl, alkynyl, amino, nitro (—NO₂), alkoxy, haloalkoxy, thioalkoxy, alkanoyl, haloalkanoyl and carbonyloxy.
R₅ can be selected from the group consisting of hydrogen, halo, hydroxy, thiol, cyano, formyl, ═O, alkyl, haloalkyl, alkenyl, alkynyl, amino, nitro, alkoxy, haloalkoxy, thioalkoxy, alkanoyl, haloalkanoyl and carbonyloxy.
R₆ and R₇ together can be ══O. Alternatively, R₆ can be selected from the group consisting of hydrogen, halo, cyano, formyl, alkyl, haloalkyl, alkenyl, alkynyl, alkanoyl and haloalkanoyl, and R₇ is independently selected from the group consisting of hydrogen, halo, hydroxy, thiol, cyano, formyl, alkyl, haloalkyl, alkenyl, alkynyl, amino, nitro, alkoxy, haloalkoxy, thioalkoxy, alkanoyl, haloalkanoyl and carbonyloxy. Alternatively, R₇ can be selected from the group consisting of hydrogen, halo, cyano, formyl, alkyl, haloalkyl, alkenyl, alkynyl, alkanoyl and haloalkanoyl, and R₆ is independently selected from the group consisting of hydrogen, halo, hydroxy, thiol, cyano, formyl, alkyl, haloalkyl, alkenyl, alkynyl, amino, nitro, alkoxy, haloalkoxy, thioalkoxy, alkanoyl, haloalkanoyl and carbonyloxy.

R₅ and R₉, independently, can be

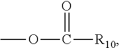

and R₁₀ is selected from the group consisting of hydrogen, halo, cyano, alkyl, haloalkyl, alkenyl and alkynyl.

Alternatively, R₅ and R₉, independently, can be

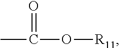

and R₁₁ is selected from the group consisting of hydrogen, halo, alkyl, haloalkyl, alkenyl and alkynyl.

Alternatively, R₅ and R₉, independently, can be

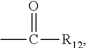

and R₁₂ is selected from the group consisting of hydrogen, halo, hydroxy, thiol, cyano, alkyl, haloalkyl, alkenyl, alkynyl, amino and nitro.

Alternatively, R₅ and R₉, independently, can be

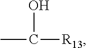

and R₁₃ is selected from the group consisting of hydrogen, halo, alkyl, haloalkyl, alkenyl and alkynyl.

Alternatively, R₈ and R₉, independently, can be selected from the group consisting of hydrogen, halo, hydroxy, thiol, cyano, formyl, ══O, alkyl, haloalkyl, alkenyl, alkynyl, amino, nitro, alkoxy, haloalkoxy, thioalkoxy, alkanoyl, haloalkanoyl and carbonyloxy.

X can be oxygen or sulfur, and Rₓ is nothing. Alternatively, X can be nitrogen, and Rₓ is selected from the group consisting of hydrogen, halo, formyl, alkyl, haloalkyl, alkenyl, alkynyl, alkanoyl, haloalkanoyl and a nitrogen protective group. Alternatively, X can be carbon, and Rₓ is selected from the group consisting of hydrogen, halo, hydroxy, thiol, cyano, formyl, ══O, alkyl, haloalkyl, alkenyl, alkynyl, amino, nitro, alkoxy, haloalkoxy, thioalkoxy, alkanoyl, haloalkanoyl and carbonyloxy.

Y can be oxygen or sulfur, and R_{y} is nothing. Alternatively, Y can be nitrogen, and R_{y} is selected from the group consisting of hydrogen, halo, formyl, alkyl, haloalkyl, alkenyl, alkynyl, alkanoyl, haloalkanoyl and a nitrogen protective group. Alternatively, Y can be carbon, and R_{y} is selected from the group consisting of hydrogen, halo, hydroxy, thiol, cyano, formyl, ══O, alkyl, haloalkyl, alkenyl, alkynyl, amino, nitro, alkoxy, haloalkoxy, thioalkoxy, alkanoyl, haloalkanoyl and carbonyloxy.

Z can be oxygen or sulfur, and R_{z} is nothing. Alternatively, Z is nitrogen, and R_{z} is selected from the group consisting of hydrogen, halo, formyl, alkyl, haloalkyl, alkenyl, alkynyl, alkanoyl, haloalkanoyl and a nitrogen protective group. Alternatively, Z can be carbon, and R_{z} is selected from hydrogen, halo, hydroxy, thiol, cyano, formyl, ══O, alkyl, haloalkyl, alkenyl, alkynyl, amino, nitro, alkoxy, haloalkoxy, thioalkoxy, alkanoyl, haloalkanoyl and carbonyloxy.

Other AHR ligands include stilbene derivatives and flavone derivatives of formula I and formula II, respectively:

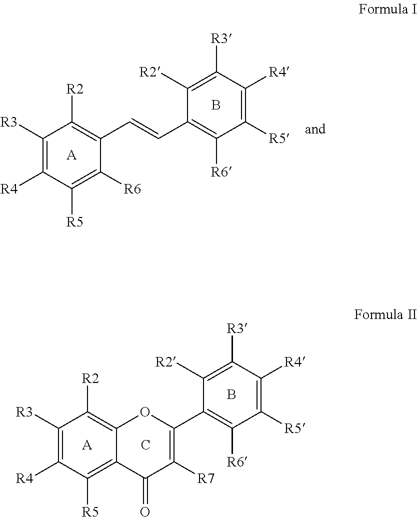

wherein R2, R3, R4, R5, R6, R7 and R2′ R3′, R4′, R5′, R6′ are identical or different (including all symmetrical derivatives) and represent H, OH, R (where R represents substituted or unsubstituted, saturated or unsaturated, linear or branched aliphatic groups containing one to thirty carbon atoms), Ac (where Ac represents substituted or unsubstituted, saturated or unsaturated, cyclic compounds, including alicyclic and heterocyclic, preferably containing three to eight atoms), Ar (where Ar represents substituted or unsubstituted, aromatic or heteroaromatic groups preferably containing five or six atoms), Cr (where Cr represents substituted or unsubstituted fused Ac and/or Ar groups, including Spiro compounds and norbornane systems, preferably containing two to five fused rings), OR, X (where X represents an halogen atom), CX₃, CHX₂, CH₂X, glucoside, galactoside, mannoside derivates, sulfate and glucuronide conjugates. Optical and geometrical isomeric derivatives of stilbene and flavone compounds are included. Among the compounds encompassed by the general formulas I and II are apigenin (4′,5,7-trihydroxyflavone), luteolin (3′,4′,5,7-tetrahydroxyflavone), tangeritin (4′,5,6,7,8-pentamethoxyflavone), diosmin (5-Hydroxy-2-(3-hydroxy-4-methoxyphenyl)-7-[(2S,3R,4S,5S,6R)-3,4,5-trihyd-roxy-6-[[(2R,3R,4R,5R,6S)-3,4,5-trihydroxy-6-methyloxan-2-yl]oxymethyl]oxa-n-2-yl]oxychromen-4-one), flavoxate (2-(1-piperidyl)ethyl 3-methyl-4-oxo-2-phenyl-chromene-8-carboxylate), piceatannol (3,4,3′,5′-tetrahydroxystilbene), oxyresveratrol (2,3′,4,5′-tetrahydroxystilbene), 4,4′-dihydroxystilbene. See US20110293537.

Nagy et al., Toxicol. Sci. 65:200-10 (2002), described a high-throughput screen useful for identifying and confirming other ligands. See also Nagy et al., Biochem. 41:861-68 (2002). In some embodiments, those ligands useful in the nanoparticle compositions are those that bind competitively with TCDD, FICZ, TA, and/or ITE.

Alternatives to AHR Ligands

In some embodiments, as an alternative or in addition to the AHR-specific ligands, the nanoparticle compositions comprise inhibitors of p38, inhibitors of Nuclear Factor kappa B (NF-kB) or Suppressor of cytokine signaling-2 (Socs2) activators.

P38 Inhibitors

A “p38 inhibitor” is any molecule (e.g., small molecules or proteins) capable of inhibiting the activity of p38 family members as determined by Western blot quantification of phosphorylated p38 levels. Examples of p38 inhibitors include SD282 (2-(6-chloro-5-((2R,5S)-4-(4-fluorobenzyl)-2,5-dimethylpiperazine-1-carbonyl)-1-methyl-1H-indol-3-yl)-N,N-dimethyl-2-oxoacetamide); 6-chloro-5-[[(2S,5R)-4-[(4-fluorophenyl)methyl]-2,5-domethyl-1-piperaziny-1]carbonyl]-N,N,1-trimethyl-.alpha.-oxo-1H-indole-3-acetamide; SKF86002 (6-(4-Fluorophenyl)-5-(4-pyridyl)-2,3-dihydroimidazo[2,1-b]-thiazole); PD169316 (4-[5-(4-fluorophenyl)-2-(4-nitrophenyl)-1H-imidazol-4-yl]-pyridine); SC68376 (2-Methyl-4-phenyl-5-(4-pyridyl)oxazole); VX702; VX745; R130823; AMG548; SCIO469; SCIO323; FR167653; MW012069ASRM; SD169; RWJ67657; ARRY797; SB203580 (4-[4-(4-fluorophenyl)-2-(4-methylsulfinylphenyl)-1H-imidazol-5-yl]pyridine); LY 2228820 (5-(2-tert-butyl-4-(4-fluorophenyl)-1H-imidazol-5-yl)-3-neopentyl-3H-imidazo[4,5-b]pyridin-2-amine dimethanesulfonate); SB202190 (4-(4-fluorophenyl)-2-(4-hydroxyphenyl)-5-(4-pyridyl)-1H-imidazole) and derivatives thereof; SB239063 (trans-1-(4-Hydroxycyclohexyl)-4-(4-fluorophenyl)-5-(2-methoxypyridimidin-4-yl)imidazole); BMS 582949)4-[[5-[(cyclopropylamino)carbonyl]-2-methylphenyl]amino]-5-methyl-N-propylpyrrolo[2,1-f][1,2,4]triazine-6-carboxamide, see US20060235020); SB220025 and derivatives thereof; PD169316; RPR200765A; SB681323 (Dilmapimod); AMG548 (2-[[(2S)-2-amino-3-phenylpropyl]amino]-3-methyl-5-(2-naphthalenyl)-6-(4-pyridinyl)-4(3H)-pyrimidinone); ARRY-797; ARRY-371797; BIRB-796 (Doramapimod, 1-(3-tert-butyl-1-p-tolyl-1H-pyrazol-5-yl)-3-(4-(2-morpholinoethoxy)naphthalen-1-yl)urea); 856553 (Losmapimod, 6-[5-(cyclopropylcarbamoyl)-3-fluoro-2-methylphenyl]-N-(2,2-dimethylpropyl)pyridine-3-carboxamide); AZD6703; KC-706; PH 797804; R1503; SC-80036; SC1O-469; SC10-323; VX-702 or VX745 (5-(2,6-dichlorophenyl)-2-(phenylthio)-6H-pyrimido[1,6-b]pyridazin-6-one); and FR167653. See, e.g., WO 2005/042502, U.S. Pat. No. 8,518,983, US20140315301, US20130028978, and US20130244262.

Also included are dominant negative mutants of p38, e.g., p38T180A, wherein the threonine at position 180 located in the DNA-binding region of p38 was mutated to alanine by point mutation; and p38Y182F, wherein the tyrosine at position 182 of p38 in human and mouse was mutated to phenylalanine by point mutation. See, e.g., US20130244262.

NF-kB Inhibitors

The NF-kB inhibitors useful in the present methods include those that act directly at the IKK complex or IkappaB phosphorylation; enhance ubiquitination or proteasomal degradation of IkappaB; inhibit nuclear translocation of NF-kappaB; or inhibit NF-kappaB DNA binding. See, e.g., Gilmore and Herscovitch, Oncogene 25:6887-6899 (2006).

Exemplary compounds include celastrol; dexamethasone; triptolide; CAY10512; helenalin; NFκB activation inhibitor II, JSH-23; andrographolide; sulfasalazine; rapamycin and rapamycin derivatives (e.g., temsirolimus and everolimus); caffeic acid phenethylester; SN50 (a cell-permeable inhibitory peptide); parthenolide; triptolide; wedelolactone; lactacystin; MG-132 [Z-Leu-Leu-Leu-H]. rocaglamide; sodium salicylate; pyrrolidinedithiocarbamic acid; substituted resorcinols, (E)-3-(4-methylphenylsulfonyl)-2-propenenitrile (Bay 11-7082); tetrahydrocurcuminoids (such as Tetrahydrocurcuminoid CG); lignans (manassantins, (+)-saucernetin, (−)-saucerneol methyl ether), sesquiterpenes (costunolide, parthenolide, celastrol, celaphanol A), diterpenes (excisanin, kamebakaurin), triterpenes (avicin, oleandrin), and polyphenols (resveratrol, epigallocatechin gallate, quercetin). See, e.g, Nam, Mini Rev Med Chem. 2006 August; 6(8):945-51; Gilmore and Herscovitch, Oncogene 25:6887-6899 (2006); and US 20130164393.

Socs2 Activators

In some embodiments, the methods include the use of a Socs2 activator, e.g., as described in US20120282646, e.g., a Socs2 protein or nucleic acid encoding a Socs2 protein. Sequences for human Socs2 protein isoforms are known in the art, e.g., GenBank Acc. Nos. NP_001257396.1, NP_001257397.1, NP_001257398.1, NP_001257399.1, NP_001257400.1, and NP_003868.1. The nucleic acid sequences encoding those protein isoforms are NM_001270467.1, NM_001270468.1, NM_001270469.1, NM_001270470.1, NM_001270471.1 and NM_003877.4.

Diabetes Autoantigens

Autoantibodies against insulin, glutamic acid decarboxylase (GAD), and other islet cell autoantigens, e.g., IGRP, ICA 512/IA-2 protein tyrosine phosphatase, ICA12, and ICA69, are frequently found in newly diagnosed diabetic subjects. Thus, type 1 diabetes autoantigens useful in the methods described herein include, e.g., preproinsulin or an immunologically active fragment thereof (e.g., insulin B-chain, A chain, C peptide or an immunologically active fragment thereof), IGRP, and other islet cell autoantigens (ICA), e.g., GAD65, islet tyrosine phosphatase ICA512/IA-2, ICA12, ICA69 or immunologically active fragments thereof. Other type 1 diabetes autoantigens include islet-specific glucose-6-phosphatase catalytic subunit-related protein (IGRP), HSP60, HSP70, carboxypeptidase H, peripherin, gangliosides (e.g., GM1-2, GM3), or immunologically active fragments thereof. Any of the type 1 diabetes autoantigens known in the art or described herein, or immunologically active fragments, analogs or derivatives thereof, are useful in the methods and compositions described herein.

Insulin, Preproinsulin, Proinsulin, and Fragments Thereof

Autoantibodies against insulin are frequently found in newly diagnosed diabetic subjects. The insulin mRNA is translated as a 110 amino acid single chain precursor called preproinsulin, and removal of its signal peptide during insertion into the endoplasmic reticulum generates proinsulin. Proinsulin consists of three domains: an amino-terminal B chain, a carboxy-terminal A chain and a connecting peptide in the middle known as the C peptide. Within the endoplasmic reticulum, proinsulin is exposed to several specific endopeptidases which excise the C peptide, thereby generating the mature form of insulin which consists of the A and B chain. Insulin and free C peptide are packaged in the Golgi into secretory granules which accumulate in the cytoplasm. The preproinsulin peptide sequence is as follows, with the B chain underlined:

(SEQ ID NO: 1)
MALWMRLLPL LALLALWGPD PAAAFVNQHL CGSHLVEALY
LVCGERGFFY TPKTRREAED LQVGQVELGG GPGAGSLQPL
ALEGSLQKRG IVEQCCTSIC SLYQLENYCN

Insulin A chain includes amino acids 90-110 of SEQ ID NO:1. B chain includes amino acids 25-54 of SEQ ID NO:1. The connecting sequence (amino acids 55-89 of SEQ ID NO:1) includes a pair of basic amino acids at either end. Proteolytic cleavage of proinsulin at these dibasic sequences liberates the insulin molecule and free C peptide, which includes amino acids 57-87 of SEQ ID NO:1. The human preproinsulin or an immunologically active fragment thereof, e.g., B chain or an immunogenic fragment thereof, e.g., amino acids 33-47 of SEQ ID NO:1 (corresponding to residues 9-23 of the B-chain), are useful as autoantigens in the methods and compositions described herein.

Engineered fragments of insulin can also be used, e.g., engineered insulin dimers as described in WO2004110373, incorporated herein by reference in its entirety.

Glutamic Acid Decarboxylase (GAD)—GAD65

Gad65 is a primary β-cell antigen involved in the autoimmune response leading to insulin dependent diabetes mellitus (Christgau et al. (1991) J Biol Chem. 266(31):21257-64). The presence of autoantibodies to GAD65 is used as a method of diagnosis of type 1 diabetes. Gad65 is a 585 amino acid protein as follows (SEQ ID NO:2).

(SEQ ID NO: 2)
MASPGSGFWS FGSEDGSGDS ENPGTARAWC QVAQKFTGGI
GNKLCALLYG DAEKPAESGG SQPPRAAARK AACACDQKPC
SCSKVDVNYA FLHATDLLPA CDGERPTLAF LQDVMNILLQ
YVVKSFDRST KVIDFHYPNE LLQEYNWELA DQPQNLEEIL
MHCQTTLKYA IKTGHPRYFN QLSTGLDMVG LAADWLTSTA
NTNMFTYEIA PVFVLLEYVT LKKMREIIGW PGGSGDGIFS
PGGAISNMYA MMIARFKMFP EVKEKGMAAL PRLIAFTSEH
SHFSLKKGAA ALGIGTDSVI LIKCDERGKM IPSDLERRIL
EAKQKGFVPF LVSATAGTTV YGAFDPLLAV ADICKKYKIW
MHVDAAWGGG LLMSRKHKWK LSGVERANSV TWNPHKMMGV
PLQCSALLVR EEGLMQNCNQ MHASYLFQQD KHYDLSYDTG
DKALQCGRHV DVFKLWLMWR AKGTTGFEAH VDKCLELAEY
LYNIIKNREG YEMVFDGKPQ HTNVCFWYIP PSLRTLEDNE
ERMSRLSKVA PVIKARMMEY GTTMVSYQPL GDKVNFFRMV
ISNPAATHQD IDFLIEEIER LGQDL

Islet Tyrosine Phosphatase IA-2

IA-2/ICA512, a member of the protein tyrosine phosphatase family, is another major autoantigen in type 1 diabetes (Lan et al. DNA Cell. Biol. 13:505-514,1994). 70% of diabetic subjects have autoantibodies to IA-2, which appear years before the development of clinical disease. The IA-2 molecule (SEQ ID NO:3, below) is 979 amino acids in length and consists of an intracellular, transmembrane, and extracellular domain (Rabin et al. (1994) J. Immunol. 152 (6), 3183-3188). Autoantibodies are typically directed to the intracellular domain, e.g., amino acids 600-979 of SEQ ID NO:3 and fragments thereof (Zhang et al. (1997) Diabetes 46:40-43; Xie et al. (1997) J. Immunol. 159:3662-3667). The amino acid sequence of IA-2 is as follows.

(SEQ ID NO: 3)
MRRPRRPGGLGGSGGLRLLLCLLLLSSRPGGCSAVSAHGCLFDRRLCSHL
EVCIQDGLFGQCQVGVGQARPLLQVTSPVLQRLQGVLRQLMSQGLSWHDD
LTQYVISQEMERIPRLRPPEPRPRDRSGLAPKRPGPAGELLLQDIPTGSA
PAAQHRLPQPPVGKGGAGASSSLSPLQAELLPPLLEHLLLPPQPPHPSLS
YEPALLQPYLFHQFGSRDGSRVSEGSPGMVSVGPLPKAEAPALFSRTASK
GIFGDHPGHSYGDLPGPSPAQLFQDSGLLYLAQELPAPSRARVPRLPEQG
SSSRAEDSPEGYEKEGLGDRGEKPASPAVQPDAALQRLAAVLAGYGVELR
QLTPEQLSTLLTLLQLLPKGAGRNPGGVVNVGADIKKTMEGPVEGRDTAE
LPARTSPMPGHPTASPTSSEVQQVPSPVSSEPPKAARPPVTPVLLEKKSP
LGQSQPTVAGQPSARPAAEEYGYIVTDQKPLSLAAGVKLLEILAEHVHMS
SGSFINISVVGPALTFRIRHNEQNLSLADVTQQAGLVKSELEAQTGLQIL
QTGVGQREEAAAVLPQTAHSTSPMRSVLLTLVALAGVAGLLVALAVALCV
RQHARQQDKERLAALGPEGAHGDTTFEYQDLCRQHMATKSLFNRAEGPPE
PSRVSSVSSQFSDAAQASPSSHSSTPSWCEEPAQANMDISTGHMILAYME
DHLRNRDRLAKEWQALCAYQAEPNTCATAQGEGNIKKNRHPDFLPYDHAR
IKLKVESSPSRSDYINASPIIEHDPRMPAYIATQGPLSHTIADFWQMVWE
SGCTVIVMLTPLVEDGVKQCDRYWPDEGASLYHVYEVNLVSEHIWCEDFL
VRSFYLKNVQTQETRTLTQFHFLSWPAEGTPASTRPLLDFRRKVNKCYRG
RSCPIIVHCSDGAGRTGTYILIDMVLNRMAKGVKEIDIAATLEHVRDQRP
GLVRSKDQFEFALTAVAEEVNAILKALPQ

ICA12

ICA 12 (Kasimiotis et al. (2000) Diabetes 49(4):555-61; Gen bank Accession No. AAD16237; SEQ ID NO:4) is one of a number of islet cell autoantigens associated with diabetes. The sequence of ICA12 is as follows.

(SEQ ID NO: 4)
MSMRSPISAQ LALDGVGTMV NCTIKSEEKK EPCHEAPQGS
ATAAEPQPGD PARASQDSAD PQAPAQGNFR GSWDCSSPEG
NGSPEPKRPG ASEAASGSQE KLDFNRNLKE VVPAIEKLLS
SDWKERFLGR NSMEAKDVKG TQESLAEKEL QLLVMIHQLS
TLRDQLLTAH SEQKNMAAML FEKQQQQMEL ARQQQEQIAK
QQQQLIQQQH KINLLQQQIQ QVNMPYVMIP AFPPSHQPLP
VTPDSQLALP IQPIPCKPVE YPLQLLHSPP APVVKRPGAM
ATHHPLQEPS QPLNLTAKPK APELPNTSSS PSLKMSSCVP
RPPSHGGPTR DLQSSPPSLP LGFLGEGDAV TKAIQDARQL
LHSHSGALDG SPNTPFRKDL ISLDSSPAKE RLEDGCVHPL
EEAMLSCDMD GSRHFPESRN SSHIKRPMNA FMVWAKDERR
KILQAFPDMH NSSISKILGS RWKSMTNQEK QPYYEEQARL
SRQHLEKYPD YKYKPRPKRT CIVEGKRLRV GEYKALMRTR
RQDARQSYVI PPQAGQVQMS SSDVLYPRAA GMPLAQPLVE
HYVPRSLDPN MPVIVNTCSL REEGEGTDDR HSVADGEMYR
YSEDEDSEGE EKSDGELVVL TD

ICA69

ICA69 is another autoantigen associated with type 1 diabetes (Pietropaolo et al. J. Clin. Invest. 1993; 92:359-371). The amino acid sequence of ICA69 is as follows.

(SEQ ID NO: 5)
MSGHKCSYPW DLQDRYAQDK SVVNKMQQRY WETKQAFIKA
TGKKEDEHVV ASDADLDAKL ELFHSIQRTC LDLSKAIVLY
QKRICFLSQE ENELGKFLRS QGFQDKTRAG KMMQATGKAL
CFSSQQRLAL RNPLCRFHQE VETFRHRAIS DTWLTVNRME
QCRTEYRGAL LWMKDVSQEL DPDLYKQMEK FRKVQTQVRL
AKKNFDKLKM DVCQKVDLLG ASRCNLLSHM LATYQTTLLH
FWEKTSHTMA AIHESFKGYQ PYEFTTLKSL QDPMKKLVEK
EEKKKINQQE STDAAVQEPS QLISLEEENQ RKESSSFKTE
DGKSILSALD KGSTHTACSG PIDELLDMKS EEGACLGPVA
GTPEPEGADK DDLLLLSEIF NASSLEEGEF SKEWAAVFGD
GQVKEPVPTM ALGEPDPKAQ TGSGFLPSQL LDQNMKDLQA
SLQEPAKAAS DLTAWFSLFA DLDPLSNPDA VGKTDKEHEL LNA

Glima38

Glima 38 is a 38 kDa islet cell membrane autoantigen which is specifically immunoprecipitated with sera from a subset of prediabetic individuals and newly diagnosed type 1 diabetic subjects. Glima 38 is an amphiphilic membrane glycoprotein, specifically expressed in islet and neuronal cell lines, and thus shares the neuroendocrine expression patterns of GAD65 and IA2 (Aanstoot et al. J. Clin. Invest. 1996 Jun. 15; 97(12):2772-2783).

Heat Shock Protein 60 (HSP60) and 70 (HSP70)

HSP60, e.g., an immunologically active fragment of HSP60, e.g., p277 (see Elias et al., Eur. J. Immunol. 1995 25(10):2851-7), can also be used as an autoantigen in the methods and compositions described herein. Other useful epitopes of HSP60 are described, e.g., in U.S. Pat. No. 6,110,746.

Similarly, HSP70, e.g., an immunologically active fragment of HSP70 (see, e.g., Abulafia-Lapid, J. Autoimmunity 20(4):313-321 (June 2003); Millar et al. Nat. Med., 9 (2003), pp. 1469-1476; Raska and Weigl, Biomed Pap Med Fac Univ Palacky Olomouc Czech Repub. 2005 December; 149(2):243-9). Other useful epitopes of HSP70 are described, e.g., in US20060089302.

Carboxypeptidase H

Carboxypeptidase H has been identified as an autoantigen, e.g., in pre-type 1 diabetes subjects (Castano et al. (1991) J. Clin. Endocrinol. Metab. 73(6):1197-201; Alcalde et al. J. Autoimmun. 1996 August; 9(4):525-8.). Therefore, carboxypeptidase H or immunologically reactive fragments thereof (e.g., the 136-amino acid fragment of carboxypeptidase-H described in Castano, supra) can be used in the methods and compositions described herein.

Peripherin

Peripherin is a 58 KDa diabetes autoantigen identified in NOD mice (Boitard et al. (1992) Proc. Natl. Acad. Sci. U.S.A. 89(1):172-6. The human peripherin sequence is shown as SEQ ID NO:6, below.

(SEQ ID NO: 6)
MSHHPSGLRA GFSSTSYRRT FGPPPSLSPG AFSYSSSSRF
SSSRLLGSAS PSSSVRLGSF RSPRAGAGAL LRLPSERLDF
SMAEALNQEF LATRSNEKQE LQELNDRFAN FIEKVRFLEQ
QNAALRGELS QARGQEPARA DQLCQQELRE LRRELELLGR
ERDRVQVERD GLAEDLAALK QRLEEETRKR EDAEHNLVLF
RKDVDDATLS RLELERKIES LMDEIEFLKK LHEEELRDLQ
VSVESQQVQQ VEVEATVKPE LTAALRDIRA QYESIAAKNL
QEAEEWYKSK YADLSDAANR NHEALRQAKQ EMNESRRQIQ
SLTCEVDGLR GTNEALLRQL RELEEQFALE AGGYQAGAAR
LEEELRQLKE EMARHLREYQ ELLNVKMALD IEIATYRKLL
EGEESRISVP VHSFASLNIK TTVPEVEPPQ DSHSRKTVLI
KTIETRNGEQ VVTESQKEQR SELDKSSAHS Y

Islet-Specific Glucose-6-Phosphatase Catalytic Subunit-Related Protein (IGRP)

IGRP, also known as glucose-6-phosphatase, catalytic 2 (G6PC2), is part of a multicomponent system that catalyzes the hydrolysis of glucose-6-phosphate, the terminal step in gluconeogenic and glycogenolytic pathways, allowing the release of glucose into the bloodstream. IGRP is found in pancreatic islets and does not exhibit phosphohydrolase activity, and is a major target of cell-mediated autoimmunity in diabetes (Jarchum et al., Clin Immunol. 2008 June; 127(3): 359-365).

There are two variant of IGRP, Isoform 1 (NP_066999.1) and Isoform 2:

| (Isoform 1, SEQ ID NO: 7) | |
|---|---|
| 1 | mdflhrngvl iiqhlqkdyr ayytflnfms nvgdprniff iyfplcfqfn qtvgtkmiwv |
| 61 | avigdwlnli fkwilfghrp ywwvqetqiy pnhsspcleq fpttcetgpg spsghamgas |
| 121 | cvwyvmvtaa lshtvcgmdk fsitlhrltw sflwsvfwli qisvcisrvf iathfphqvi |
| 181 | lgviggmlva eafehtpgiq taslgtylkt nlflflfavg fylllrvlni dllwsvpiak |
| 241 | kwcanpdwih idttpfaglv rnlgvlfglg fainsemfll scrggnnytl sfrllcalts |
| 301 | ltilqlyhfl qiptheehlf yvlsfcksas ipltvvafip ysvhmlmkqs gkksq |
| (Isoform 2, SEQ ID NO: 8) | |
| 1 | mdflhrngvl iiqhlqkdyr ayytflnfms nvgdprniff iyfplcfqfn qtvgtkmiwv |
| 61 | avigdwlnli fkwilfghrp ywwvqetqiy pnhsspcleq fpttcetgpg spsghamgas |
| 121 | cvwyvmvtaa lshtvcgmdk fsitlhrhag grgl |

Antigenic fragments of IGRP include IGRP 265-273 (VLFGLGFAI; SEQ ID NO:9); IGRP 211-219 (NLFLFLFAV; SEQ ID NO:10); IGRP 215-223 (FLFAVGFYL; SEQ ID NO:11); and IGRP 222-230 (YLLLRVLNI; SEQ ID NO:12).

Gangliosides

Gangliosides can also be useful autoantigens in the methods and compositions described herein. Gangliosides are sialic acid-containing glycolipids which are formed by a hydrophobic portion, the ceramide, and a hydrophilic part, i.e. the oligosaccharide chain. Gangliosides are expressed, inter alia, in cytosol membranes of secretory granules of pancreatic islets. Auto-antibodies to gangliosides have been described in type 1 diabetes, e.g., GM1-2 ganglioside is an islet autoantigen in diabetes autoimmunity and is expressed by human native β cells (Dotta et al. Diabetes. 1996 September; 45(9):1193-6). Gangliosides GT3, GD3 and GM-1 are also the target of autoantibodies associated with autoimmune diabetes (reviewed in Dionisi et al. Ann. Ist. Super. Sanita. 1997; 33(3):433-5). Ganglioside GM3 participates in the pathological conditions of insulin resistance (Tagami et al. J Biol Chem 277:3085-3092 (2002)).

Antibodies

In some embodiments, the nanoparticles also include antibodies to selectively target a cell. The term “antibody,” as used herein, refers to full-length, two-chain immunoglobulin molecules and antigen-binding portions and fragments thereof, including synthetic variants. A typical full-length antibody includes two heavy (H) chain variable regions (abbreviated herein as VH), and two light (L) chain variable regions (abbreviated herein as VL). The term “antigen-binding fragment” of an antibody, as used herein, refers to one or more fragments of a full-length antibody that retain the ability to specifically bind to a target. Examples of antigen-binding fragments include, but are not limited to: (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., Nature 341:544-546 (1989)), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. Science 242:423-426 (1988); and Huston et al. Proc. Natl. Acad. Sci. USA 85:5879-5883 (1988)). Such single chain antibodies are also encompassed within the term “antigen-binding fragment.”

Production of antibodies and antibody fragments is well documented in the field. See, e.g., Harlow and Lane, 1988. Antibodies, A Laboratory Manual. Cold Spring Harbor, N.Y.: Cold Spring Harbor Laboratory. For example, Jones et al., Nature 321: 522-525 (1986), which discloses replacing the CDRs of a human antibody with those from a mouse antibody. Marx, Science 229:455-456 (1985), discusses chimeric antibodies having mouse variable regions and human constant regions. Rodwell, Nature 342:99-100 (1989), discusses lower molecular weight recognition elements derived from antibody CDR information. Clackson, Br. J. Rheumatol. 3052: 36-39 (1991), discusses genetically engineered monoclonal antibodies, including Fv fragment derivatives, single chain antibodies, fusion proteins chimeric antibodies and humanized rodent antibodies. Reichman et al., Nature 332: 323-327 (1988) discloses a human antibody on which rat hypervariable regions have been grafted. Verhoeyen, et al., Science 239: 1534-1536 (1988), teaches grafting of a mouse antigen binding site onto a human antibody.

In the methods described herein, it would be desirable to target the compounds to T cells, B cells, dendritic cells, and/or macrophages, therefore antibodies selective for one or more of those cell types can be used. For example, for T cells, anti-CXCR4, anti-CD28, anti-CD8, anti-CTLA4, or anti-CD3 antibodies can be used; for B cells, antibodies to CD20, CD19, or to B-cell receptors can be used; for dendritic cell targeting, exemplary antibodies to CD11c, DEC205, MHC class I or class II, CD80, or CD86 can be used; for macrophages, exemplary antibodies to CD11b, MHC class I or class II, CD80, or CD86 can be used. Other suitable antibodies are known in the art.

Biocompatible Nanoparticles

The nanoparticles useful in the methods and compositions described herein are made of materials that are (i) biocompatible, i.e., do not cause a significant adverse reaction in a living animal when used in pharmaceutically relevant amounts; (ii) feature functional groups to which the binding moiety can be covalently attached, (iii) exhibit low non-specific binding of interactive moieties to the nanoparticle, and (iv) are stable in solution, i.e., the nanoparticles do not precipitate. The nanoparticles can be monodisperse (a single crystal of a material, e.g., a metal, per nanoparticle) or polydisperse (a plurality of crystals, e.g., 2, 3, or 4, per nanoparticle).

A number of biocompatible nanoparticles are known in the art, e.g., organic or inorganic nanoparticles. Liposomes, dendrimers, carbon nanomaterials and polymeric micelles are examples of organic nanoparticles. Quantum dots can also be used. Inorganic nanoparticles include metallic nanoparticle, e.g., Au, Ni, Pt and TiO2 nanoparticles. Magnetic nanoparticles can also be used, e.g., spherical nanocrystals of 10-20 nm with a Fe2+ and/or Fe3+ core surrounded by dextran or PEG molecules. In some embodiments, colloidal gold nanoparticles are used, e.g., as described in Qian et al., Nat. Biotechnol. 26(1):83-90 (2008); U.S. Pat. Nos. 7,060,121; 7,232,474; and U.S. P.G. Pub. No. 2008/0166706. Suitable nanoparticles, and methods for constructing and using multifunctional nanoparticles, are discussed in e.g., Sanvicens and Marco, Trends Biotech., 26(8): 425-433 (2008).

In all embodiments, the nanoparticles are attached (linked) to the AHR ligands described herein via a functional groups. In some embodiments, the nanoparticles are associated with a polymer that includes the functional groups, and also serves to keep the metal oxides dispersed from each other. The polymer can be a synthetic polymer, such as, but not limited to, polyethylene glycol or silane, natural polymers, or derivatives of either synthetic or natural polymers or a combination of these. Useful polymers are hydrophilic. In some embodiments, the polymer “coating” is not a continuous film around the magnetic metal oxide, but is a “mesh” or “cloud” of extended polymer chains attached to and surrounding the metal oxide. The polymer can comprise polysaccharides and derivatives, including dextran, pullanan, carboxydextran, carboxmethyl dextran, and/or reduced carboxymethyl dextran. The metal oxide can be a collection of one or more crystals that contact each other, or that are individually entrapped or surrounded by the polymer.

In other embodiments, the nanoparticles are associated with non-polymeric functional group compositions. Methods are known to synthesize stabilized, functionalized nanoparticles without associated polymers, which are also within the scope of this invention. Such methods are described, for example, in Halbreich et al., Biochimie, 80 (5-6):379-90, 1998.

In some embodiments, the nanoparticles have an overall size of less than about 1-100 nm, e.g., about 25-75 nm, e.g., about 40-60 nm, or about 50-60 nm in diameter. The polymer component in some embodiments can be in the form of a coating, e.g., about 5 to 20 nm thick or more. The overall size of the nanoparticles is about 15 to 200 nm, e.g., about 20 to 100 nm, about 40 to 60 nm; or about 60 nm.

Synthesis of Nanoparticles

There are varieties of ways that the nanoparticles can be prepared, but in all methods, the result must be a nanoparticle with functional groups that can be used to link the nanoparticle to the binding moiety.

For example, the autoantigens and AHR ligands can be linked to the metal oxide through covalent attachment to a functionalized polymer or to non-polymeric surface-functionalized metal oxides. In the latter method, the nanoparticles can be synthesized according to a version of the method of Albrecht et al., Biochimie, 80 (5-6): 379-90, 1998. Dimercapto-succinic acid is coupled to the nanoparticle and provides a carboxyl functional group. By functionalized is meant the presence of amino or carboxyl or other reactive groups that can be used to attach desired moieties to the nanoparticles, e.g., the AHR ligands described herein or antibodies.

In another embodiment, the AHR ligands are attached to the nanoparticles via a functionalized polymer associated with the nanoparticle. In some embodiments, the polymer is hydrophilic. In a specific embodiment, the conjugates are made using oligonucleotides that have terminal amino, sulfhydryl, or phosphate groups, and superparamagnetic iron oxide nanoparticles bearing amino or carboxy groups on a hydrophilic polymer. There are several methods for synthesizing carboxy and amino derivatized-nanoparticles. Methods for synthesizing functionalized, coated nanoparticles are discussed in further detail below.

Carboxy functionalized nanoparticles can be made, for example, according to the method of Gorman (see WO 00/61191). Carboxy-functionalized nanoparticles can also be made from polysaccharide coated nanoparticles by reaction with bromo or chloroacetic acid in strong base to attach carboxyl groups. In addition, carboxy-functionalized particles can be made from amino-functionalized nanoparticles by converting amino to carboxy groups by the use of reagents such as succinic anhydride or maleic anhydride.

Nanoparticle size can be controlled by adjusting reaction conditions, for example, by varying temperature as described in U.S. Pat. No. 5,262,176. Uniform particle size materials can also be made by fractionating the particles using centrifugation, ultrafiltration, or gel filtration, as described, for example in U.S. Pat. No. 5,492,814.

Nanoparticles can also be treated with periodate to form aldehyde groups. The aldehyde-containing nanoparticles can then be reacted with a diamine (e.g., ethylene diamine or hexanediamine), which will form a Schiff base, followed by reduction with sodium borohydride or sodium cyanoborohydride.

Dextran-coated nanoparticles can also be made and cross-linked, e.g., with epichlorohydrin. The addition of ammonia will react with epoxy groups to generate amine groups, see Hogemann et al., Bioconjug. Chem. 2000. 11(6):941-6, and Josephson et al., Bioconjug. Chem., 1999, 10(2):186-91.

Carboxy-functionalized nanoparticles can be converted to amino-functionalized magnetic particles by the use of water-soluble carbodiimides and diamines such as ethylene diamine or hexane diamine.

Avidin or streptavidin can be attached to nanoparticles for use with a biotinylated binding moiety, such as an oligonucleotide or polypeptide. See e.g., Shen et al., Bioconjug. Chem., 1996, 7(3):311-6. Similarly, biotin can be attached to a nanoparticle for use with an avidin-labeled binding moiety.

In all of these methods, low molecular weight compounds can be separated from the nanoparticles by ultra-filtration, dialysis, magnetic separation, or other means. The unreacted AHR ligands can be separated from the ligand-nanoparticle conjugates, e.g., by size exclusion chromatography.

In some embodiments, colloidal gold nanoparticles are made using methods known in the art, e.g., as described in Qian et al., Nat. Biotechnol. 26(1):83-90 (2008); U.S. Pat. Nos. 7,060,121; 7,232,474; and U.S. P.G. Pub. No. 2008/0166706.

In some embodiments, the nanoparticles are pegylated, e.g., as described in U.S. Pat. Nos. 7,291,598; 5,145,684; 6,270,806; 7,348,030, and others.

### Methods of Treatment

As described herein, a subject who is at risk of developing T1D, or in the early stages of developing T1D (i.e., before complete loss of insulin-secreting pancreatic islet cells, wherein the subject can still make their own insulin), can be treated by increasing the number of Treg cells and/or the activity of Treg cells using a therapeutically effective amount of nanoparticle that co-delivers one or more diabetes autoantigens and one or more transcription factor ligands (e.g., TCDD, tryptamine (TA), and/or 2-(1′H-indole-3′-carbonyl)-thiazole-4-carboxylic acid methyl ester (ITE)) that are capable of promoting an increase in the expression and/or activity of Foxp3, and thereby promoting an increase in the number or activity of Treg cells in vitro and/or in vivo and inhibiting or stopping the autoimmune destruction of the subject's pancreas.

In some embodiments, the methods include administering a composition comprising a nanoparticle linked to one or more diabetes autoantigens and a ligand that activates the AHR receptor. In some embodiments, the composition is co-administered with one or more inhibitors of its degradation, e.g., tryptamine together with a monoamine oxidase inhibitor (MAOI), e.g., hydrazines such as isocarboxazid; nialamide; phenelzine; or hydracarbazine; or tranylcypromine. The inhibitor can be administered in the same or in a separate composition. Thus the invention also includes compositions comprising nanoparticles comprising a diabetes autoantigen and tryptamine and an inhibitor of tryptamine degradation, e.g., a MAOI, e.g., tranylcypromine.

Alternatively or in addition, a population of cells capable of differentiation into Treg cells (e.g., naïve T cells and/or CD4⁺CD62 ligand⁺ T cells) can be contacted with a nanoparticle linked to one or more diabetes autoantigens and a transcription factor ligand capable of promoting increase in Foxp3 expression and/or activity (e.g., TCDD, TA, ITE) in vitro, thereby effectively promoting an increase in the number of Treg cells in the population. Alternatively or in addition, a population of cells containing Treg cells (e.g., isolated Treg cells (e.g., 100%) or a population of cells containing at least 20, 30, 40, 50, 60, 70, 80, 90, 95, or 99% Treg cells) can be contacted with nanoparticles linked to one or more diabetes autoantigens and a transcription factor ligand capable of promoting an increase in Foxp3 expression and/or activity (e.g., TCDD, TA, or ITE), thereby effectively promoting an increase in the activity of the Treg cells in the population. One or more cells from these populations can then be administered to the subject.

Subject Selection

The compositions and methods described herein are of particular use for treating a subject (e.g., a human) that would benefit from therapeutic immunomodulation (e.g., a subject in need of a suppressed immune response) to treat, prevent, or reduce the risk of developing T1D. The methods include selecting a subject in need of treatment and administering to the subject one or more of the compositions described herein. A subject in need of treatment can be identified, e.g., by their medical practitioner.

In some embodiments, the methods include determining presence and/or levels of autoantibodies to an autoantigen specific for the disease, e.g., the presence and/or levels of autoantibodies to a diabetes autoantigen described herein, e.g., to proinsulin, GAD, and/or ICA, e.g., ICA 512/IA-2, ICA12, and ICA69. The results can be used to determine a subject's likelihood or risk of developing the disease; subjects can be selected for treatment using a method described herein based on the presence and/or levels of autoantibodies. See, e.g., Yu et al., Proc Natl Acad Sci USA 97, 1701-1706 (2000); Mamchak et al., Diabetes 61, 1490-1499 (2012); Quintana et al., Proc Natl Acad Sci USA 101 Suppl 2, 14615-14621 (2004).

Alternatively or in addition, the subject can be identified based on the presence of a family history of T1D, e.g., a first degree relative (parent or sibling) diagnosed with T1D. Alternatively or in addition, the subject can be identified based on the presence of one or more symptoms of early stage T1D, e.g., increased or extreme thirst; frequent urination; sugar in urine; bedwetting in children who previously didn't wet the bed during the night; extreme hunger; sudden or unintended weight loss; irritability and other mood changes; fatigue and weakness; blurred vision; fruity, sweet, or wine-like odor on breath; heavy, labored breathing; and in females, a vaginal yeast infection. Risk factors for T1D include family history; the presence of T1D predisposing genes; geography (distance from the equator increases risk); and age (4 to 7 years and 10 to 14 years being the highest-risk groups). In some embodiments, subjects who can be treated using the methods described herein are in the so-called “honeymoon period’ (i.e., partial remission) of type 1 diabetes mellitus, which is characterized by reduced insulin requirements while good glycemic control is maintained (e.g., a period with insulin requirements of less than 0.5 U/kg/day and hemoglobin A1c (HbA1c) level of less or equal to 6%; see, e.g., Abdul-Rasoul et al., Pediatr Diabetes. 2006 April; 7(2):101-7). Subjects who can be treated using the methods described herein retain some residual beta cell function, or have had or are about to have a transplant of functioning beta cells, e.g., autologous stem cells such as bone marrow stem cells, induced pluripotent stem cells, hematopoietic stem cells; umbilical cord stem cells, or beta cells derived therefrom (see, e.g., Mesples et al., Med Sci Monit. 2013 Oct. 14; 19:852-7; Kanafi et al., Cytotherapy. 2013 October; 15(10):1228-36; Fujikura et al. Endocr J. 2013; 60(6):697-708; Li et al., J Diabetes. 2012 December; 4(4):332-7; Baas et al., J Immunol. 2014 Nov. 1; 193(9):4696-703; Kessell et al., Clin Transl Gastroenterol. 2015 Jan. 29; 6:e73; Wilson et al., Ann Surg. 2014 October; 260(4):659-65; discussion 665-7).

Validation of Treatment/Monitoring Treatment Efficacy

During and/or following treatment, a subject can be assessed at one or more time points, for example, using methods known in the art for assessing severity of the specific autoimmune disease or its symptoms, to determine the effectiveness of the treatment. In some embodiments, levels of autoantibodies to an autoantigen specific for the disease can also be monitored, e.g., levels of autoantibodies to a diabetes autoantigen; a decrease (e.g., a significant decrease) in levels of autoantibodies would indicate a positive response, i.e., indicating that the treatment is successful; see, e.g., Mesples et al., Med Sci Monit. 2013 Oct. 14; 19:852-7). Treatment can then be continued without modification, modified to improve the progress or outcome (e.g., increase dosage levels, frequency of administration, the amount of the pharmaceutical composition, and/or change the mode of administration), or stopped.

Administration

A therapeutically effective amount of one or more of the compositions described herein can be administered by standard methods, for example, by one or more routes of administration, e.g., by one or more of the routes of administration currently approved by the United States Food and Drug Administration (FDA; see, for example world wide web address fda.gov/cder/dsm/DRG/drg00301.htm), e.g., orally, topically, mucosally, intravenously or intramuscularly.

Pharmaceutical Formulations

A therapeutically effective amount of the nanoparticles described herein can be incorporated into pharmaceutical compositions suitable for administration to a subject, e.g., a human. Such compositions typically include the composition and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances are known. Except insofar as any conventional media or agent is incompatible with the active compound, such media can be used in the compositions of the invention. Supplementary active compounds can also be incorporated into the compositions, e.g., an inhibitor of degradation of the ligand.

A pharmaceutical composition can be formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the composition (e.g., an agent described herein) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL™ (sodium carboxymethyl starch), or corn starch; a lubricant such as magnesium stearate or STEROTES™; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration. In one aspect, the pharmaceutical compositions can be included as a part of a kit.

Generally the dosage used to administer a pharmaceutical compositions facilitates an intended purpose for prophylaxis and/or treatment without undesirable side effects, such as toxicity, irritation or allergic response. Although individual needs may vary, the determination of optimal ranges for effective amounts of formulations is within the skill of the art. Human doses can readily be extrapolated from animal studies (Katocs et al., Chapter 27 In: “Remington's Pharmaceutical Sciences”, 18th Ed., Gennaro, ed., Mack Publishing Co., Easton, Pa., 1990). Generally, the dosage required to provide an effective amount of a formulation, which can be adjusted by one skilled in the art, will vary depending on several factors, including the age, health, physical condition, weight, type and extent of the disease or disorder of the recipient, frequency of treatment, the nature of concurrent therapy, if required, and the nature and scope of the desired effect(s) (Nies et al., Chapter 3, In: Goodman & Gilman's “The Pharmacological Basis of Therapeutics”, 9th Ed., Hardman et al., eds., McGraw-Hill, New York, N.Y., 1996).

### Kits

The present invention also includes kits, e.g., for use in the methods described herein. In some embodiments the kit comprise one or more doses of a composition described herein. The composition, shape, and type of dosage form for the induction regimen and maintenance regimen may vary depending on a subjects requirements. For example, dosage form may be a parenteral dosage form, an oral dosage form, a delayed or controlled release dosage form, a topical, and a mucosal dosage form, including any combination thereof.

In a particular embodiment, a kit can contain one or more of the following in a package or container: (1) one or more doses of a composition described herein; (2) one or more pharmaceutically acceptable adjuvants or excipients (e.g., a pharmaceutically acceptable salt, solvate, hydrate, stereoisomer, and clathrate); (3) one or more vehicles for administration of the dose; (5) instructions for administration. Embodiments in which two or more, including all, of the components (1)-(5), are found in the same container can also be used.

When a kit is supplied, the different components of the compositions included can be packaged in separate containers and admixed immediately before use. Such packaging of the components separately can permit long term storage without loosing the active components' functions. When more than one bioactive agent is included in a particular kit, the bioactive agents may be (1) packaged separately and admixed separately with appropriate (similar of different, but compatible) adjuvants or excipients immediately before use, (2) packaged together and admixed together immediately before use, or (3) packaged separately and admixed together immediately before use. If the chosen compounds will remain stable after admixing, the compounds may be admixed at a time before use other than immediately before use, including, for example, minutes, hours, days, months, years, and at the time of manufacture.

The compositions included in particular kits of the present invention can be supplied in containers of any sort such that the life of the different components are optimally preserved and are not adsorbed or altered by the materials of the container. Suitable materials for these containers may include, for example, glass, organic polymers (e.g., polycarbonate and polystyrene), ceramic, metal (e.g., aluminum), an alloy, or any other material typically employed to hold similar reagents. Exemplary containers may include, without limitation, test tubes, vials, flasks, bottles, syringes, and the like.

As stated above, the kits can also be supplied with instructional materials. These instructions may be printed and/or may be supplied, without limitation, as an electronic-readable medium, such as a floppy disc, a CD-ROM, a DVD, a Zip disc, a video cassette, an audiotape, and a flash memory device. Alternatively, instructions may be published on a internet web site or may be distributed to the user as an electronic mail.

### EXAMPLES

The invention is further described in the following examples, which do not limit the scope of the invention described in the claims.

Materials and Methods

The following materials and methods were used in these Examples.

Mice and Reagents

NOD and BDC2.5 transgenic mice⁴¹ were purchased from The Jackson Laboratories (Bar Harbor, Me., USA) and kept in a pathogen-free facility at the Harvard Institutes of Medicine. For the induction of Cyclophosphamide Accelerated Diabetes (CAD), 4 mg of Cyclophosphamide monohydrate (Sigma) was injected i.p twice, 7 days apart. All experiments were carried out in accordance with the guidelines of the standing committee of animals at Harvard Medical School.

Patient Samples

Heparinized venous blood was drawn from an individual from The University of Massachusetts Adult Diabetes Clinic with Internal Review Board approval. The subject was a 58 year old female bearing HLA-DRB1*0404 with T1D for 50 years.

Human T-Cell Clone

The 325GAD T cell clone was a kind gift from Dr. Helena Reijonen (Benaroya Research Institute at Virginia Mason University, Seattle, Wash. The T cell clones was derived from the peripheral blood of a subject with T1D by sorting and expansion of CD4+ T cells binding an HLA DRB1*04:04 tetramer loaded with hGAD65_{555-567(557F→I)}. The substituted peptide stabilizes binding to the tetramer: the T cell clone recognizes the native peptide (NFFRMVISNPAAT) and the substituted peptide in the context of HLA-DRB1*04:01 and DRB1*04:04. The native peptide was used in all experiments here.

Nanoparticle Preparation

NPs were produced using ultrapure water, 60-nm Tannic Acid-stabilized gold particles at a concentration of 2.6×10¹⁰ particles per milliliter (Ted Pella Inc.), mPEG-SH (MW5000 kDa) (Nektar Therapeutics), ITE (Tocris Bioscience, Ellisville, Mo., USA), MIMO peptide (MEVGWYRSPFSRVVHLYRNGK, Cat. #62756, AnaSpec, Inc.) and Insulin protein. Freshly prepared solutions of ITE (3.5 mM), insulin or MIMO (1 mg/ml) were added dropwise to a rapidly mixing gold colloid at a 1:6 ITE solution/colloid volume ratio, which facilitates even distributions of the molecules on the gold particle surface⁵¹. After 30 min incubation at room temperature, mPEG-SH (10 mM) was added drop wise to the colloids, with a minimum ratio of 30,000 PEG-SH molecules per 60-nm gold particle. This surface coverage has been shown to result in a complete PEG monolayer on the gold particle surface, and stabilizes gold colloids against aggregation under various conditions⁵¹. Moreover, it has been reported that the addition of 10- to 20-fold excess PEG-SH does not result in any changes in colloid stability or in the thickness of the polymer coating layer⁵¹. After an additional 30 min incubation at room temperature, the colloids will be pelleted by centrifugation and re-suspended in ultrapure water, and characterized by UV-visible spectroscopy and Transmission Electron Microscopy as described⁵¹.

Protein Quantification

The incorporation of MIMO peptide or Insulin onto the NPs was assessed using the fluorescence-based peptide quantification kit LavaPep (Fluorotechnics).

Luciferase Reporter Assays

293 cells were transfected using Fugene HD (Roche) and the cells were analyzed after 24h with the dual luciferase assay kit (New England Biolabs, Ipswich, Mass.). Tk-Renilla was used for standardization.

FACS

For intracellular cytokine staining cells were stimulated in culture medium containing PMA (50 ng/ml) (Sigma-Aldrich), ionomycin (1 □g/ml) (Calbiochem, San Diego, Calif., USA) and GolgiStop (BD Biosciences) for 4 h. After staining of surface markers, cells were fixed and permeabilized as described and incubated with cytokine-specific antibodies (1:100) at 25° C. for 30 min.

Purification of Splenic DCs

DCs were purified from the spleens of naïve NOD mice using CD11c⁺ magnetic beads according to the manufacturer's instructions (Miltenyi, Auburn, Calif., USA). Cells were incubated with NP in the presence or absence of LPS (100 ng/ml) and 48 h later the cells were used to stimulate BDC2.5+ CD4+ T cells.

Generation of Bone Marrow-Derived DCs (BMDCs)

To generate bone marrow-derived DC cells, bone marrow cells were isolated from the femurs of naïve NOD mice and cultured for 7 days in the presence of IL-4 (long/ml) and GM-CSF (20 ng/ml). On day 7 cells were purified with CD11c⁺ magnetic beads (Miltenyi, USA).

Generation of Monocyte-Derived Dendritic Cells (hDCs)

Heparinized venous blood was ficolled by standard methods and peripheral blood mononuclear cells (PBMC) plated at 5×10⁶ cells/ml in HL-1 media supplemented with 2 mM L-glutamine, 5 mM HEPES, and 100 U/ml penicillin and 100 μg/ml streptomycin, 0.1 mM each non-essential amino acids, 1 mM sodium pyruvate (all from Lonza), and 5% heat-inactivated human male AB serum (Omega Scientific), used for all experiments, for 3 hours and then non-adherent cells were removed. rhIL-4 (5 ng/ml) and rhGM-CSF (100 ng/ml) were added and after 5 days harvested cells were either used as immature DC a or matured overnight with rhTNFa (50 ng/ml), rhIL-1b (10 ng/ml) and LPS (100 ng/ml) (cytokines from R&D Systems; LPS from Sigma-Aldrich) and used as mature DC.

Mouse T-Cell Differentiation In Vitro

BDC2.5+ CD4+ T cells were activated with BMDC or splenic DCs at a 3:1 (100,000:30,000) T-cell-to-DC ratio, and activated with MIMO (20 μg/mL) as described (11).

T-Cell Proliferation and Cytokine Production

BDC2.5+ CD4+ T cells were cultured with BMDC or DCs for 72 h. During the last 16 h, cells were pulsed with 1 mCi of [3H]thymidine (PerkinElmer, Waltham, Mass., USA) followed by harvesting on glass fiber filters and analysis of incorporated [3H]thymidine in a beta-counter (1450 Microbeta, Trilux, PerkinElmer). Culture supernatants were collected after 48 h after and cytokine concentration was determined by ELISA using antibodies for IFNγ, IL-17 from BD Biosciences.

Human T-Cell Differentiation In Vitro

Immature or mature DC were plated in round bottom 96 well plates (CoStar) at 10,000/well and allowed to adhere for 4 hours. DCs were then treated with freshly prepared NP at the indicated concentrations overnight. DCs were washed in wells with PBS twice and then the 325 T cell clone was added at 30,000 cells/well in HL-1 media and incubated for 48 hours. Secreted gIFN was analyzed by ELISA (BD BioSciences).

Real Time PCR (qPCR)

RNA was extracted from cells using RNA Easy Mini Kit (Qiagen, Valencia, Calif., USA), cDNA was prepared as recommended, and real-time PCR was performed using an ABI7500 cycler (Applied Biosystems, Foster City, Calif., USA). All values were expressed as fold increase or decrease relative to the expression of gapdh.

Transmission Electron Microscopy

DC-incubated NPs were fixed in the dish for at least 1 h at room temperature with 2.5% (vol/vol) glutaraldehyde, 1.25% (vol/vol) paraformaldehyde, and 0.03% picric acid in 0.1M sodium cacodylate buffer (pH 7.4). The cells were then postfixed for 30 min in 1% OsO4/1.5% (wt/vol) KFeCN6, washed in water three times, and incubated in 1% aqueous uranyl acetate for 30 min followed by two washes in water and subsequent dehydration in grades of alcohol [5 min each; 50%, 70%, 95% (vol/vol), twice at 100%]. Cells were removed from the dish in propylene oxide, pelleted at 1,000 Å˜g for 3 min, and infiltrated for 2 h in a 1:1 mixture of propylene oxide and TAAB Epon (Marivac). The samples were then embedded in TAAB Epon and polymerized at 60° C. for 48 h. Ultrathin sections (approximately 60 nm) were cut on a Reichert Ultracut-S microtome, picked up onto copper grids stained with lead citrate, and examined in TecnaiG2 Spirit BioTWIN, and images were recorded with an AMT 2 k CCD camera.

Histology

Collected pancreata were fixed in 4% paraformaldehyde, cut and stained by standard hematoxylin and eosin, and the average degree of insulitis was assessed over 20 islets scored per pancreas. Each islet was classified as clear, if no infiltrate was detected; mildly infiltrated, if peri-insulitis or an intra-islet infiltrate occupied less than 25% of the islet; or infiltrated or heavily infiltrated, if 25-50%, or more than 50% of the islet was occupied by inflammatory cells, respectively.

BMDC Transfer Model

Bone marrow-derived DCs were generated as described above. On day 7, NPs were added to the cells and 24 h later cells were purified with CD11c⁺ magnetic beads (Miltenyi, USA). DCs (1-2×10⁶ per mouse) were then extensively washed and transferred i.v. into 8 weeks female NOD recipient mice, 4 times, once every 4 days. Glucose levels were measured in blood weekly. Mice with glycaemia higher than 200 mg/dl were considered diabetic.

Treg Transfer Model

6 weeks NOD donor mice were treated i.p, weekly, with 6 ug of NPs. 1 month later, regulatory T cells from those mice were purified using magnetic beads (CD4+ CD25+ Regulatory T cell Kit, Miltenyi Biotec). 5×10⁵ CD4+ CD25+ T cells were transferred i.v. into 6 weeks NOD recipient mice. Glucose levels were measured in blood weekly. Mice with glycaemia above 200 mg/dl were considered diabetic.

Gene Expression Analysis (Nanostring)

Nanostring nCounter technology (nanostring.com) allows expression analysis of multiple genes from a single sample⁵⁴. We customized a multiplexed target profiling of 146 inflammation- and immune-related transcripts and used in accordance with the manufacturer's protocol (Nanostring, USA). This combination of genes and their differential expression in vivo in DCs allowed us to interrogate immune-related pathways using the Expander (Expression Analyzer and Displayer) pathway analysis during EAE.

Antigen Microarrays

553 antigens were spotted onto Epoxy slides (TeleChem, Sunnyvale, Calif., USA) as described⁵⁵. The microarrays were blocked with 1% bovine serum albumin, and incubated with a 1:100 dilution of the test serum in blocking buffer. The arrays were then washed and incubated with goat anti-mouse IgG Cy3-conjugated detection antibodies (Jackson ImmunoResearch Labs, West Grove, Pa., USA). Antigen reactivity was defined by the mean intensity of binding to the replicates of that antigen on the microarray. Raw data were normalized and analyzed using the GeneSpring software (Silicon Genetics, Redwood City, Calif., USA).

Chromatin Immunoprecipitation (ChIP)

Cells were cross-linked with 1% paraformaldehyde and lysed with the appropriate lysis buffer (1% SDS, 10 mM EDTA, 50 mM Tris-HCl, pH 8.1) containing 1× protease inhibitor cocktail (Roche Molecular Biochemicals, USA). Chromatin was sheared by sonication and supernatants were collected after centrifugation and diluted in buffer (1% Triton X-100, 2 mM EDTA, 150 mM NaCl, 20 mM Tris-HCl, pH 8.1). Five mg of antibody was prebound for a minimum of 4 h to protein A and protein G Dynal magnetic beads (Invitrogen, USA) and washed three times with ice-cold PBS plus 5% BSA, and then added to the diluted chromatin and immunoprecipitated overnight. The magnetic bead-chromatin complexes were then washed 3 times in RIPA buffer (50 mM HEPES [pH 7.6], 1 mM EDTA, 0.7% Na deoxycholate, 1% NP-40, 0.5 M LiCl) followed by 2 times with TE buffer. Immunoprecipitated chromatin was then extracted with 1% SDS, M NaHCO₃ and heated at 65° C. for at least 6 h to reverse the paraformaldehyde crosslinking. DNA fragments were purified with a QIAquick DNA purification Kit (Qiagen, USA) and analyzed using SYBR green real time PCR (Takara Bio Inc., USA). We used the following antibodies for ChIP: socs2 antibody (Cat. #2779, Cell Signaling Technology, Inc., USA). The following primer pairs were used:

AhR (XRE-1):
for: 5′-GGAATGGAGCGGACAGGA-3′,
rev: 5′-GGAATGGAGCGGACAGGA-3′;
AhR (XRE-2):
for: 5′-ATGAGTCAACACGTCCCAGA-3′,
rev: 5′-CTGCACACTCTCGTTTTGGG-3′;
AhR (XRE-3):
for: 5′-TGGCAAAGTCTCTCGCAGA-3′,
rev: 5′-TGCTCGGGGTTAAATGGTAC-3′.

Western Blot

DCs and BMDCs were lysed with the appropriate amount of lysis buffer (Cell fractioning Kit; Cat. #9038, Cell Signaling Transduction) and cytoplasmatic and nuclear fraction were saved for protein quantification (Cat. #23235, Thermo scientific). Lysates of DCs were resolved by electrophoresis through 4-12% Bis-Tris Nupage gels (Invitrogen, USA) and were transferred onto PVDF membranes (Millipore). Then, membranes were blocked with 5% milk for 1 h and probed with the following antibodies at 4 degrees, shacking overnight: anti-TRAF6 (Cat. #ab33915, abcam), Socs2 (Cat. #2779, Cell Signaling Transduction), NFκB p65 (Cat. #8242, Cell Signaling Transduction), phospho-38 MAPK (Thr180/Tyr182) (Cat. #9211, Cell Signaling Transduction), p38 MAPK (Cat. #9212, Cell Signaling Transduction), phospo-p44/42 MAPK (Thr202/Tyr204) (Cat. #4376, Cell Signaling Transduction), p44/42 MAPK (Cat. #9102, Cell Signaling Transduction), GAPDH (Cat. #5174, Cell Signaling Transduction), anti-Histone H3 antibody (Cat. #07-690, Millipore). The next day membranes were washed with TBS-Tween and incubated with Anti-rabbit IgG HRP-linked antibody (Cat, #7074, Cell Signaling Transduction) for 1 h at room temperature. Blots were developed with SuperSignal West Femto Maximum Sensitivity Substrate as suggested by the manufacturer (Pierce).

siRNA Knockdown

BMDCs were generated as described in this manuscript and at day 7 were transfected with SMARTpool: ON-TARGETplus Socs2 siRNA (Cat. # L-044410-01-0005, Dharmacon) using GeneSilencer siRNA Transfection Reagent (Cat. #T5000, Genlantis) following the manufacturers protocol. After 72h, BMDCs were used for in vitro experiments.

Generation of NPs Containing β-Cell Antigens and ITE

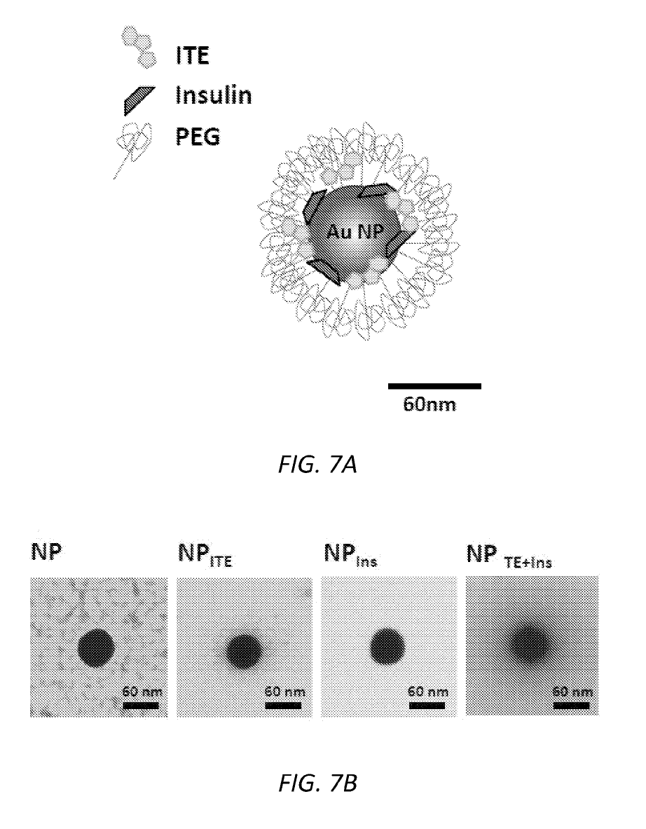

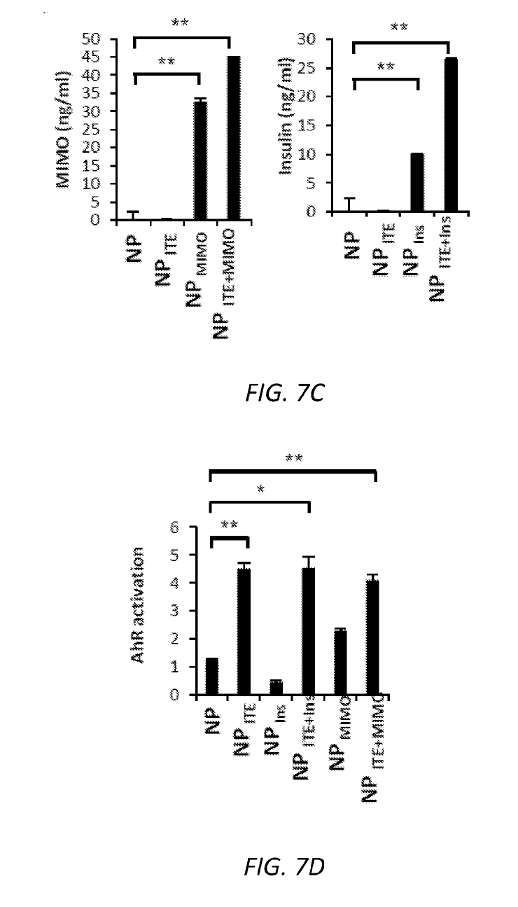

Insulin harbors epitopes targeted by diabetogenic and regulatory CD4+ and CD8+ T cells^{12, 37-39} and has been identified as an initiating autoantigen in T1 D⁴⁰. Thus, we constructed NPs containing the tolerogenic AhR ligand 2-(1′H-indole-3′-carbonyl)-thiazole-4-carboxylic acid methyl ester (ITE) and β-cell related peptide or recombinant antigens such as insulin (NP_{ITE+Ins}) or a mimotope peptide (MIMO) that activates the diabetogenic CD4⁺ T cell clone BDC-2.5 (NP_{ITE+MIMO})(Katz et al., Cell 74, 1089-1100 (1993)). We used 60 nm gold NPs to construct 4 different types of NPs: (i) unloaded NPs (NP), (ii) NPs loaded with ITE (NP_{ITE}), (iii) NPs loaded with antigen, e.g., Insulin (NP_{Ins}), (iv) NPs loaded with ITE and Insulin (NP_{ITE+Ins}) (FIG. 7A). In some studies we also used NPs loaded with the MIMO peptide recognized by the diabetogenic CD4+ T cell clone BDC-2.5⁴¹ (NP_{MIMO} and NP_{ITE+MIMO}). NP solubility and stability was improved by adding a layer of thiol-poly-ethylene glycol (PEG).

Transmission electron microscopy (TEM) studies showed that NPs in the different groups present round morphology and a diameter of approximately 60 nm (FIG. 78). Moreover, quantification studies determined that the loading of the NPs with ITE did not decrease the amount of insulin or MIMO incorporated to NPs (FIG. 7C). Conversely, ITE-containing NPs (NP_{ITE}, NP_{ITE+Ins}, NP_{ITE+MIMO}) showed comparable abilities to activate an AhR-responsive luciferase reporter, suggesting that the incorporation of insulin or MIMO did not interfere with the loading of ITE (FIG. 7D).

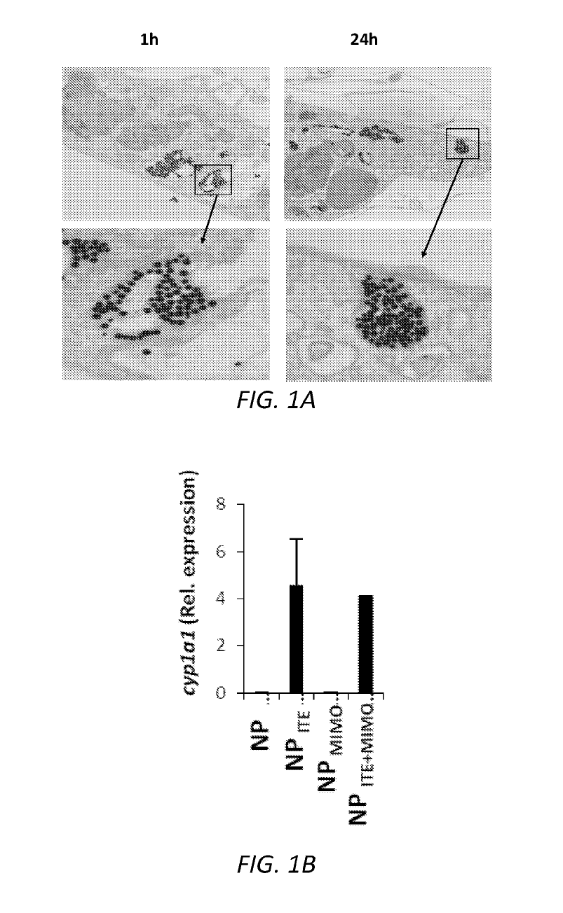

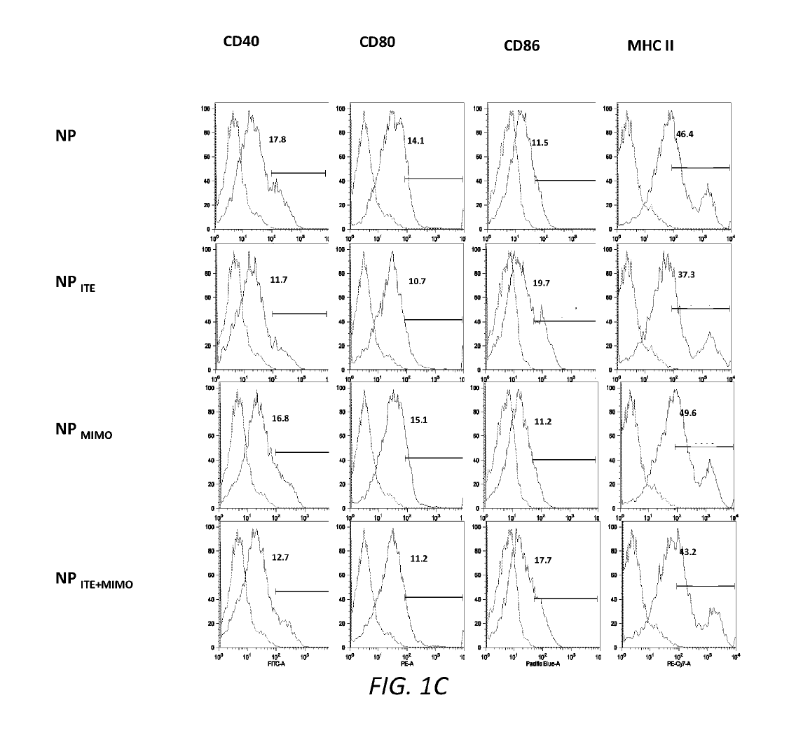

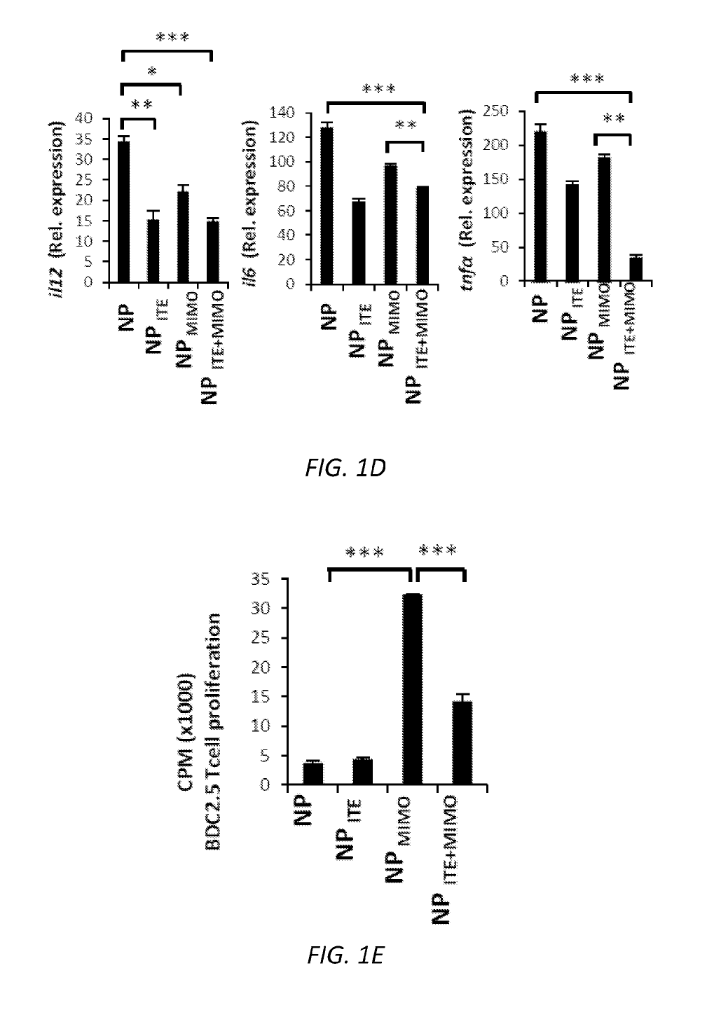

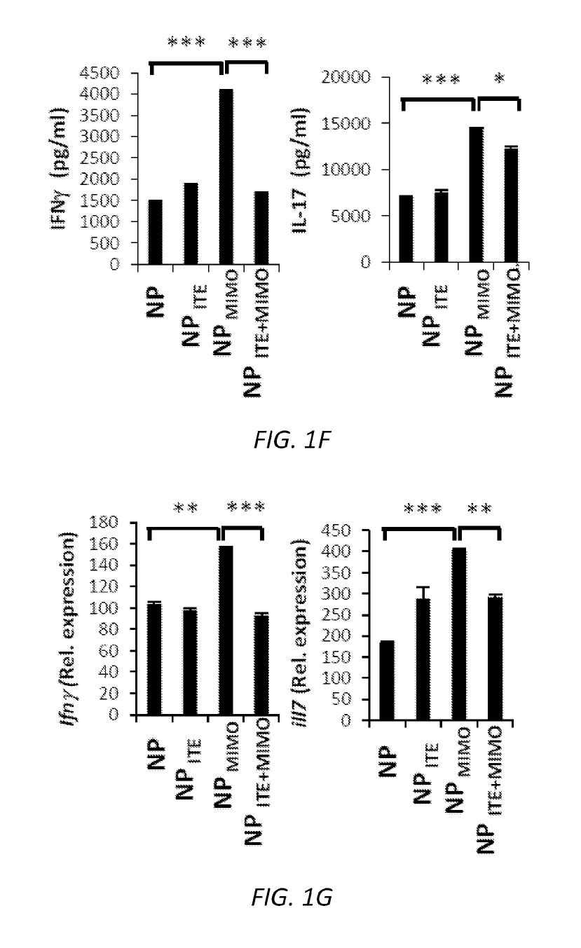

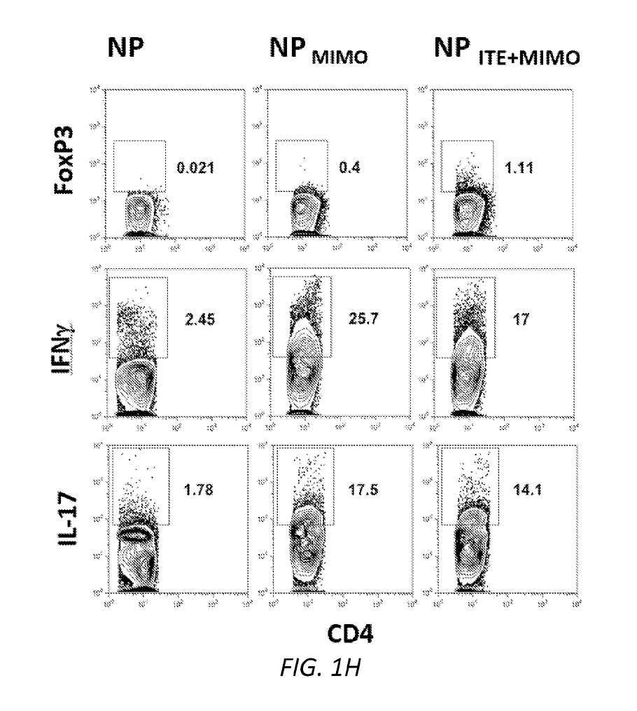

NP_{ITE+MIMO} induces tolerogenic DCs DCs control T-cell activation and polarization in vivo⁴²⁻⁴⁴ and AhR signaling has been shown to affect the antigen presenting cell (APC) function of DCs⁴²⁻⁴⁵. Thus, we studied the effects of NPs on NOD DCs. We found that NPs were uptaken within 1 hr of in vitro co-incubation with splenic DCs (FIG. 1A). Moreover, the expression of cyp1a1, which is transcriptionally controlled by AhR, was up-regulated by ITE-containing NPs (NP_{ITE} and NP_{ITE+MIMO}) but not in response to NP alone or NP_{MIMO} (FIG. 1B). Similar results were observed with bone marrow-derived DCs (BMDCs).

DCs control T-cell activation and differentiation through the expression of co-stimulatory molecules and the production of polarizing cytokines⁴²⁻⁴⁵. To study the effects of NPs on DC function we activated splenic DCs in vitro with the toll-like receptor 4 (TLR4) agonist E. coli lipopolysaccharide (LPS). We found that NP_{ITE} and NP_{ITE+MIMO} down-regulated the expression of the major histocompatibility complex class II (MHC II) and of the co-stimulatory molecules CD40 and CD80, while they up-regulated CD86 expression (FIG. 1C). NP_{ITE} and NP_{ITE+MIMO} also decreased the expression of il2a and il6 which promote Th1 and Th17 polarization, respectively (FIG. 1D). Conversely, NP_{ITE+MIMO} increased the expression of Il10, but did not alter Idol expression in DC activated with LPS.

To investigate the effects of NPs on T-cell differentiation we used transgenic BDC2.5 T cells which harbor a TCR reactive with β-cell antigens and MIMO. Splenic NOD DCs activated with LPS in the presence of NPs were co-incubated with naive CD4+ BDC2.5 T cells and T cell activation and differentiation was analyzed. We found that NP_{MIMO}-treated DCs induced the proliferation of BDC2.5 T cells (FIG. 1E) and the production of IFNγ and IL-17 in the absence of exogenously added MIMO (FIG. 1F-G), suggesting that the MIMO antigen in the NPs is delivered to the DCs and presented to T cells. In agreement with a tolerogenic role of AhR. signaling in DCs, NP_{ITE+MIMO}-treated DCs induced a lower response in terms of proliferation, IFNγ and IL-17 production by BDC2.5 T cells (FIG. 1E-G), Indeed, BDC2.5 T cells stimulated with NP_{ITE+MIMO}-treated DCs showed an increased expression of FoxP3 (FIG. 1H), resulting in higher FoxP3+/IL-17+ or FoxP3+/IFNγ+ T cell ratios (FIG. 1I). Taken together, these results suggest that AhR-targeting NPs such as NP_{ITE+MIMO} induce a tolerogenic phenotype in DCs.

NP_{ITE+Ins} Administration Arrests T1D in NOD Mice

The balance between effector and regulatory T cells is thought to play an important role in T1D development. Based on the effects of NP_{ITE+MIMO} on DCs and their ability to differentiate T cells in vitro, we studied the effects of NPs carrying ITE and insulin (NP_{ITE+Ins}) in the development of spontaneous T1D in NOD mice. In preliminary studies using the cyclophosphamide-accelerated model of diabetes (CAD) (Quintana et al., Journal of immunology (Baltimore, Md.: 1950) 169, 6030-6035 (2002), Quintana et al., Proceedings of the National Academy of Sciences of the United States of America 101 Suppl 2, 14615-14621 (2004)) we compared the effects of NP oar T1D development. NPs were administered weekly (6 ug per mouse) starting 1 week before CAD induction in 8 weeks old (wo) naïve NOD mice as described. NPITE+Ins administration suppressed the development of T1D 4 weeks after CAD induction, no significant effects were detected following treatment with NPIns; a non-significant trend towards T1D amelioration was observed in NPIns-treated mice. Hence, in follow up studies we focused on the effects of NPITE+Ins on spontaneous NOD T1D.

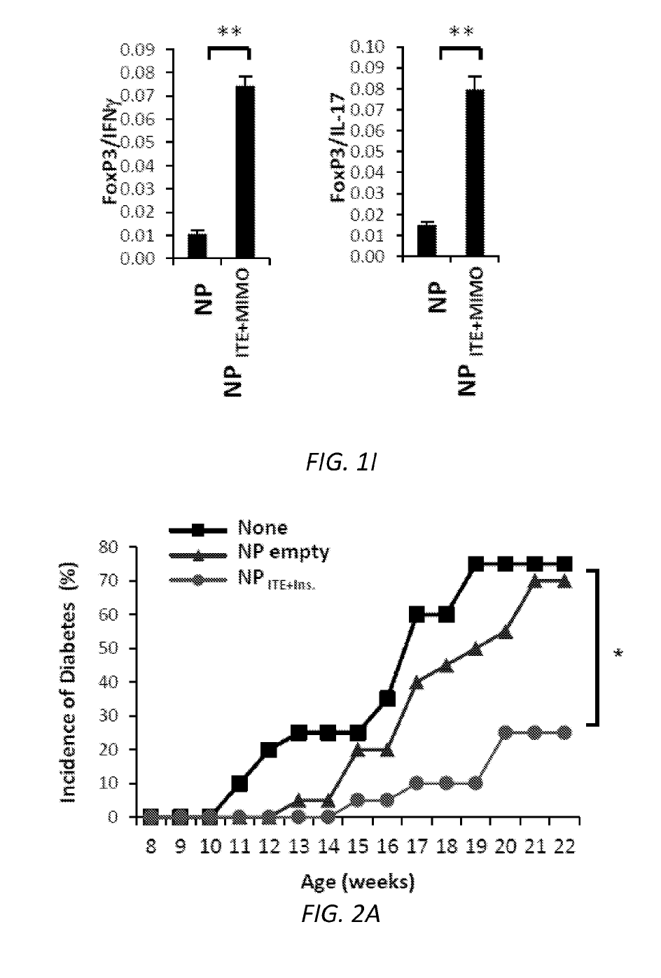

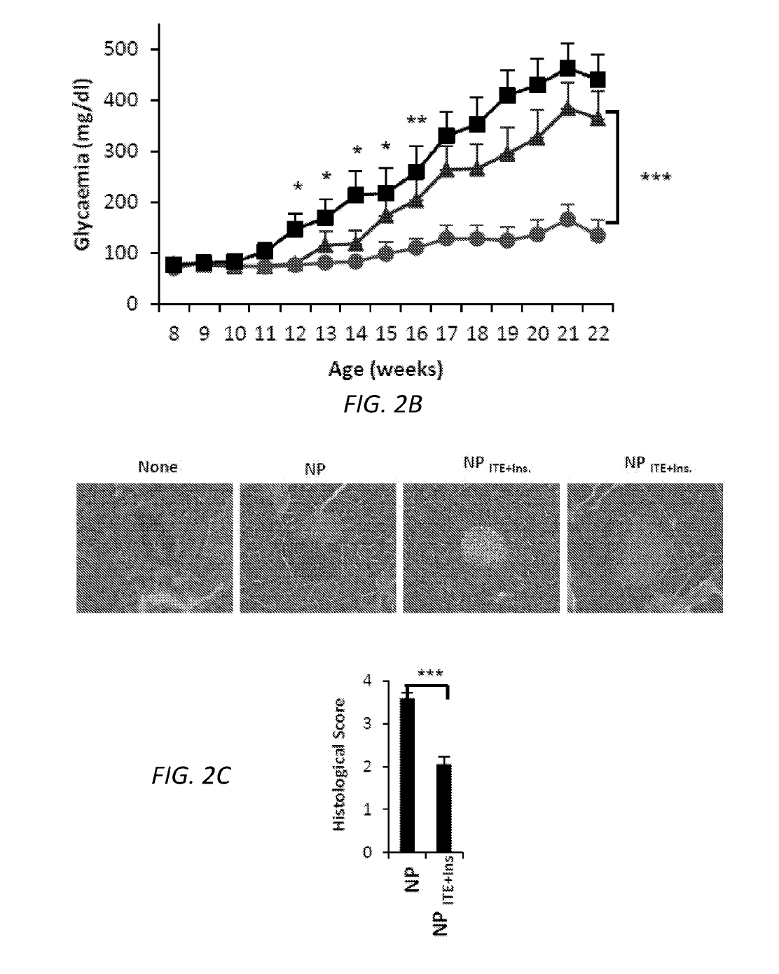

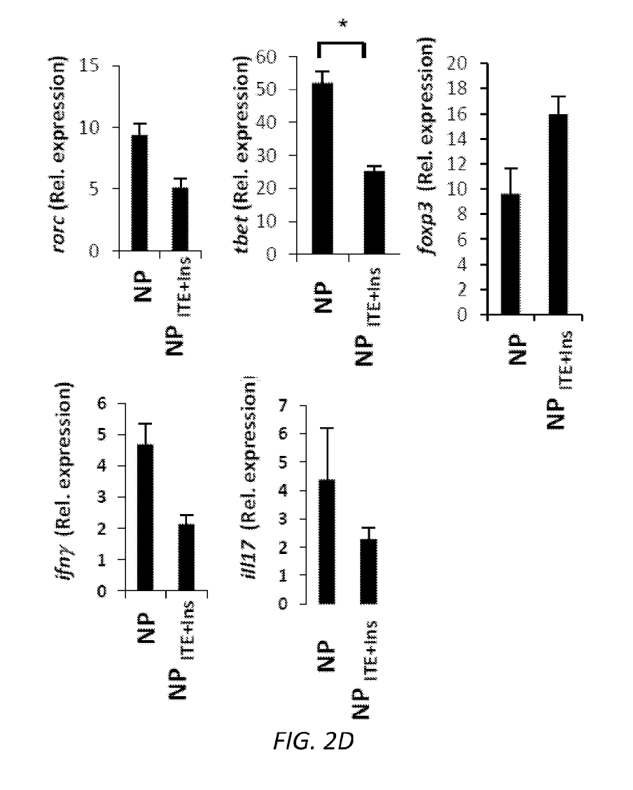

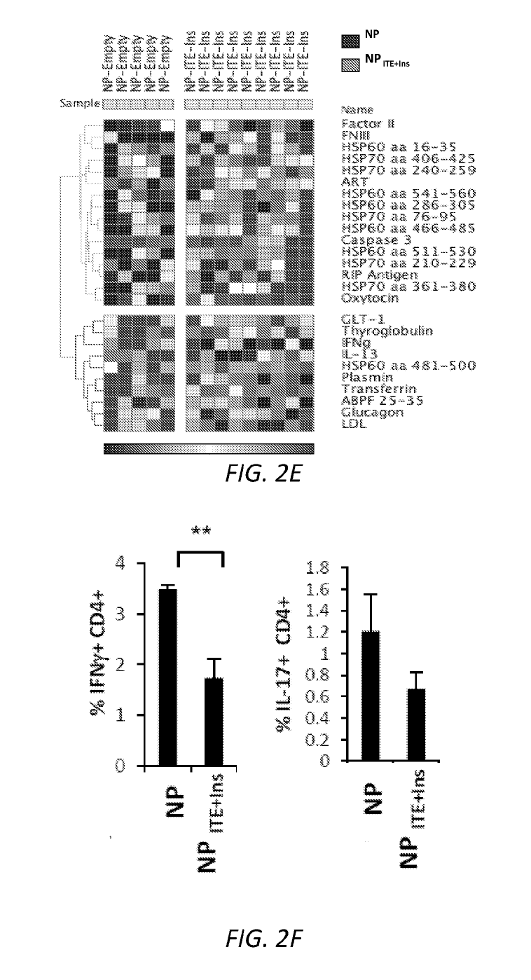

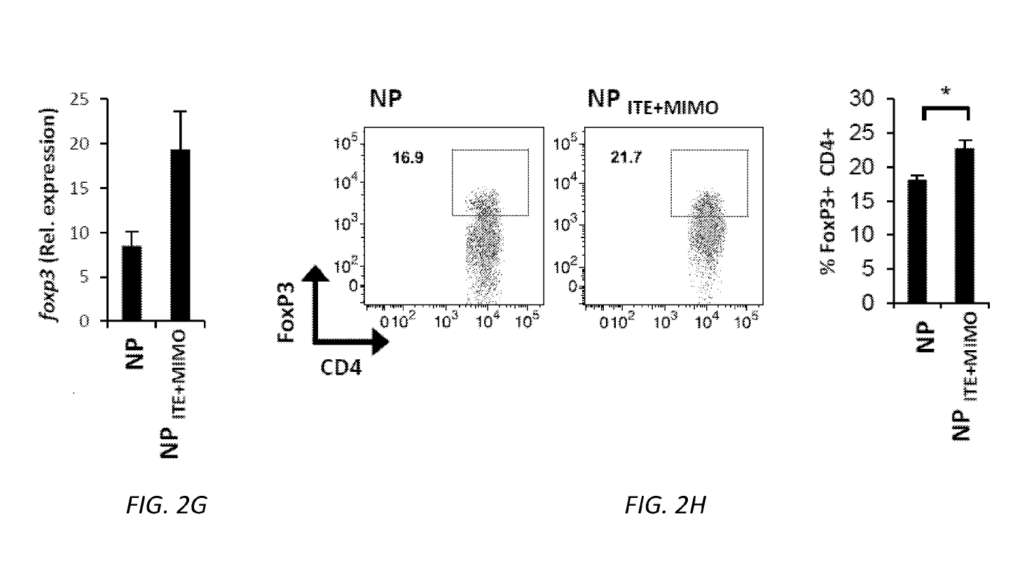

Naïve 8 week old (wo) female NOD mice were treated weekly (6 ug per mouse) with NPs and the spontaneous development of T1D was monitored. We found that NP_{ITE-Ins} administration arrested T1D development as determined by blood glucose levels and the histological analysis of pancreas samples (FIGS. 2A-C).

β-cell specific effector T cells drive T1D immune pathogenesis¹⁻⁶. In agreement with the arrest of T1D development, we detected decreased expression of tbx21 and rorc, transcription factors involved in the differentiation of Th1 and Th17 cells and decreased expression of ifng and il17 in T cells isolated from the pancreatic lymph nodes of NP_{ITE+Ins}-treated mice. Conversely, foxp3 expression was increased (FIG. 2D).

Autoantibodies targeting pancreatic antigens are detectable in T1D subjects and can be used to monitor the diabetogenic immune response⁴⁶. Indeed, serum antibody signatures detectable with antigen microarrays have been linked to the development of T1D in NOD mice⁴⁷. Thus, we analyzed the effect of NPs on the autoantibody repertoire of NOD mice. We found that treatment with NP_{ITE+Ins} modified the IgM and IgG autoantibody response of 22 wo NOD mice (FIG. 2E). Taken together, these data suggest that NP_{ITE+Ins} administration abrogates the development of spontaneous T1D.

NP_{ITE+MIMO} Controls the Diabetogenic T-Cell Response

To investigate the effects of NPs on the β-cell specific T-cell response in vivo we used NOD mice expressing a transgenic MIMO-specific BDC2.5 TCR receptor isolated from a diabetogenic T-cell clone⁴¹. We found that the administration of NP_{ITE+MIMO} to BDC2.5 NOD decreased the frequency of IFNγ+ and IL-17+ CD4+ T cells in pancreatic lymph nodes (FIG. 2F). This decrease was concomitant with an increase in the frequency of pancreatic FoxP3+ Tregs (FIGS. 2G,H). Indeed, the passive transfer CD4+ CD25+ Tregs from NP_{ITE+Ins}-treated mice suppressed cyclophosphamide accelerated diabetes in 6 wo NOD mice. Taken together, these data suggest that NP_{ITE+Ins} limits the diabetogenic T-cell response.

We then investigated the effects of NP_{ITE+MIMO} on DCs in vivo. Treatment with NP_{ITE+MIMO} did not affect the recruitment to the pancreas of classic DCs linked to T-cell activation. However, NP_{ITE+MIMO} administration up-regulated the expression of the AhR-responsive gene Cyp1a1 in DCs. Moreover, following activation with LPS ex vivo, DCs isolated from NP_{ITE+MIMO}-treated mice showed decreased expression of Il6 and Il12a, suggesting that NP_{ITE+MIMO} induces tolerogenic DCs in vivo.

NP_{ITE+MIMO} Induces Tolerogenic DCs In Vivo

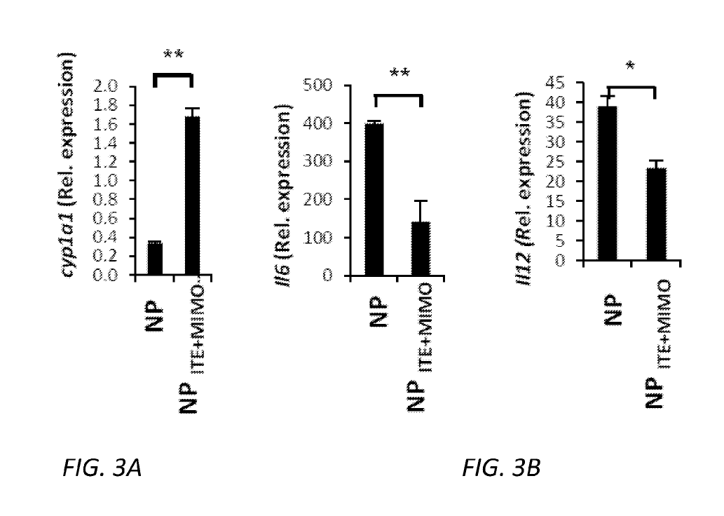

β-cell death in pre-diabetic NOD mice is associated to the recruitment of innate immune cells to the pancreas and T1D initiation⁴⁸. However, we found that NP_{ITE+Ins} decreased the recruitment of macrophages, DCs and B cells to the pancreas (FIG. 3A).

To evaluate the functional effects of NP_{ITE+MIMO} on DCs in vivo. NP_{ITE+MIMO} administration up-regulated the expression of the AhR-responsive gene cyp1a1 (FIG. 3B). Moreover, following activation with LPS ex vivo, DCs isolated from NP_{ITE+MIMO}-treated mice showed decreased the expression of il6 and il12a, suggesting that NP_{ITE+MIMO} induces tolerogenic DCs in vivo.

To study the relevance of these tolerogenic DCs for the arrest of T1D by NP_{ITE+MIMO} we carried out transfer experiments using NP-loaded DCs. BMDCs were incubated for 24h with NPs, washed and injected intravenously into 6 wo naive NOD mice. Treatment was repeated 3 additional times, once every 4 days, and the development of spontaneous T1D was monitored. We found that treatment with NP_{ITE+Ins}-loaded DCs reduced the development of spontaneous NOD T1D: 40% of the NOD recipients treated with BMDCs loaded with empty NP developed diabetes by the age of 22 weeks as opposed to 10% in the NP_{ITE+Ins} group (P<0.001, n=20 mice/group, 2 experiments) as well as a reduction on the glucose levels in bloodF. The arrest of T1D development by NP_{ITE+Ins}-loaded DCs was associated with an increase in the frequency of FoxP3+ Tregs, concomitant with a decrease in the frequency of IFNγ+CD4+ T cells. Taken together, these results suggest that administration of NP_{ITE+Ins} induces tolerogenic DCs that suppress spontaneous NOD T1D.

NP_{ITE+MIMO} Down-Regulate NF-κB Signaling in DCs

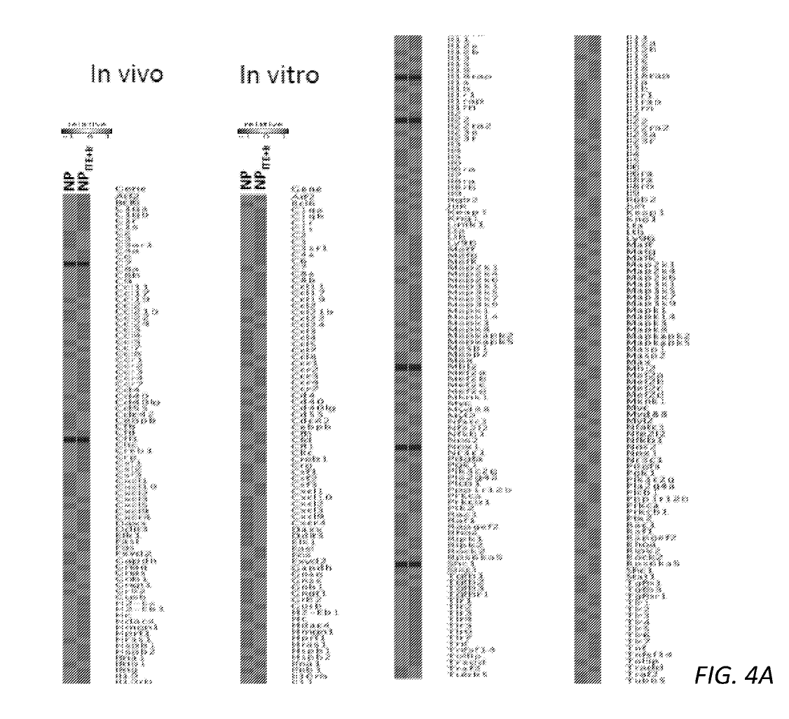

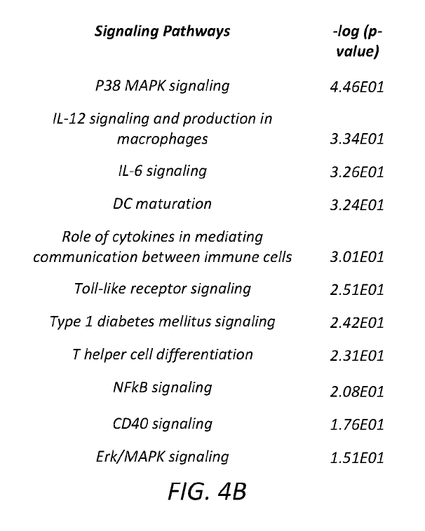

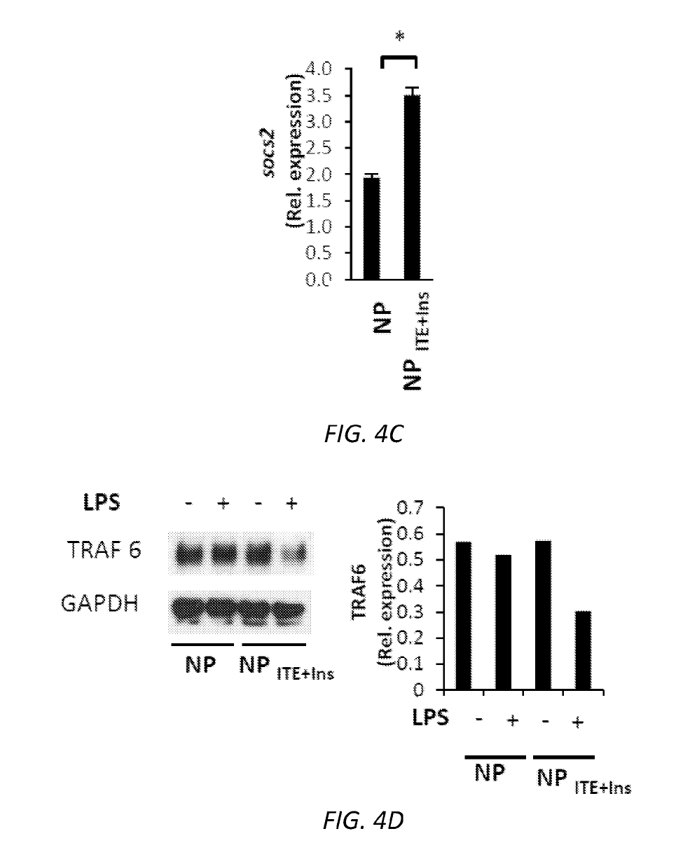

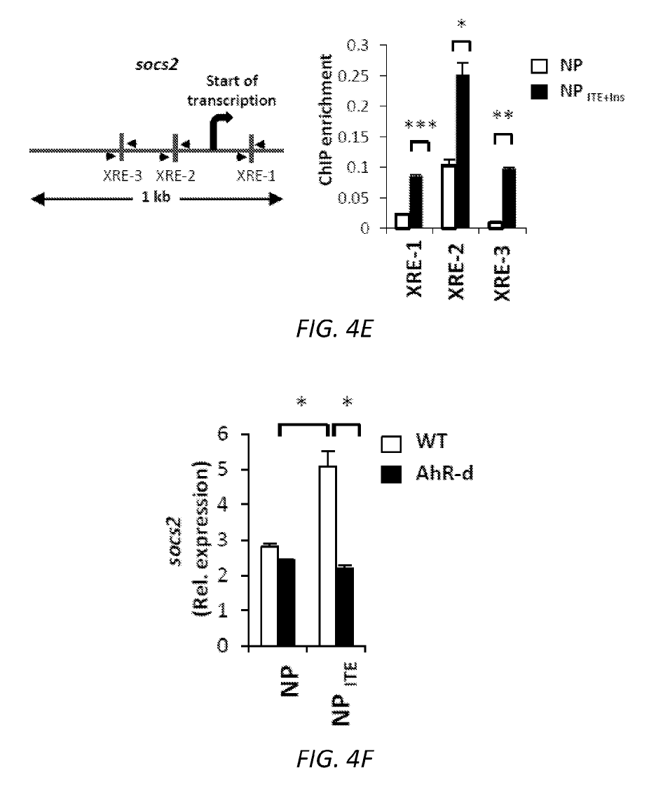

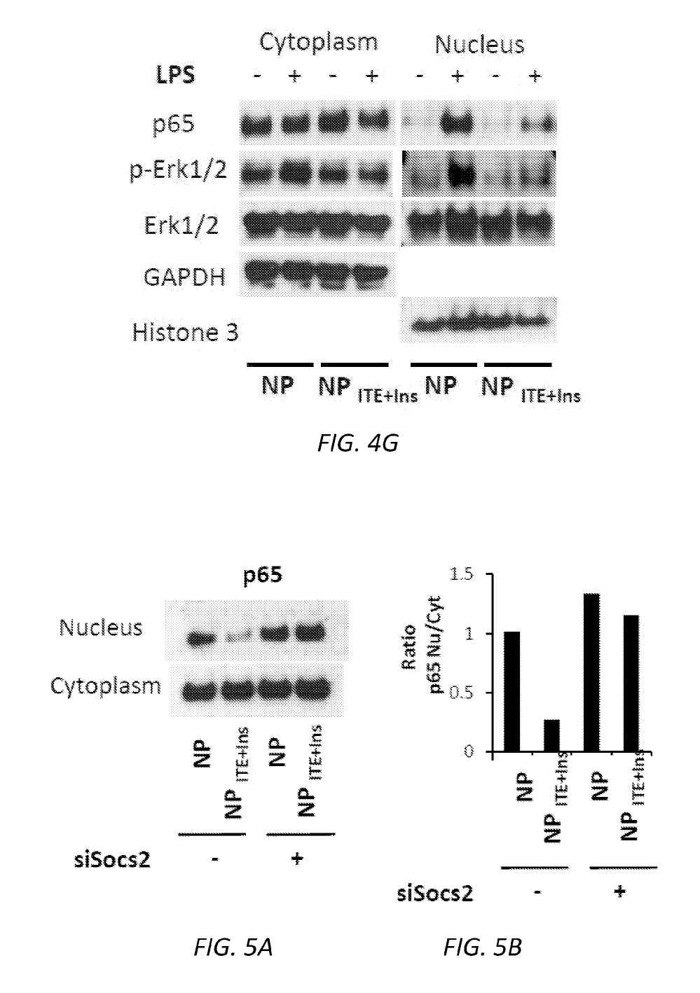

To investigate the mechanisms involved in the tolerogenic effect of NP_{ITE+Ins} we studied the transcriptional effects of NPs on splenic DCs. The analysis of the transcriptional response of splenic DCs to NP_{ITE+Ins} with Ingenuity Pathway Analysis (IPA) detected significant effects on the expression of genes associated with DC activation and maturation, the production of pro-inflammatory mediators and molecules linked to T1D pathogenesis (FIG. 4A). Based on their reported roles during inflammation, we focused on p38 MAPK, ERK MAPK and NF-κB signaling pathways.

Our transcriptional profiling experiments suggested that NP_{ITE+Ins} affects the suppressor of cytokine signaling 2 (Socs2) and the TNF receptor associated factor 6 (TRAF6) expression. Indeed, Socs2 has been shown to inhibit NFkB, p38 and ERK1/2 signaling through the inhibition of TRAF6⁵². Moreover, the AhR ligand TCDD has been previously reported to induce SOCS2 expression in B cells⁵³. Thus, we investigated the regulation of SOCS2 expression in DCs by AhR. We observed that incubation of DCs with NPs containing ITE but not NP alone, increased the expression of Socs2 (FIG. 4C), but not Socs1 or Socs3. These observations were concomitant with a reduced expression of TRAF6 in DCs treated with NP_{ITE+Ins} (FIG. 4D). A bioinformatics analysis of the socs2 promoter identified 3 potential AhR binding sites (xenobiotic responsive elements; XRE-1, XRE-2 and XRE-3) (FIG. 4E). Indeed, chromatin immunoprecipitation studies of DCs activated with LPS in the presence of NPs detected a significant recruitment of AhR to the socs2 locus in response to NP_{ITE+Ins} treatment (FIG. 4E). These data suggest that activation of AhR by NP_{ITE+Ins} induces Socs2 expression.

To validate these findings we studied the effects of NP_{ITE+Ins} on the activation and expression of p38 MAPK, ERK MAPK and NF-κB p65 in DCs (FIG. 4F). We found that treatment of splenic DCs with NP_{ITE+Ins} in the presence of LPS had no effect on p38 or ERK1/2 activation. However, we detected a significant reduction of NF-κB p65 activation and translocation to the nucleus. Taken together, these data suggest that NP_{ITE+Ins} modulates DC activation and function through the SOCS2-dependent silencing of NF-κB signaling.

The suppressor of cytokine signaling 2 (SOCS2) interferes with NF-κB, p38 MAPK and ERK1/2 signaling through the inhibition of the TNF receptor associated factor 6 (TRAF6)⁴⁹. Our transcriptional profiling studies suggested that NP_{ITE+Ins} affects Socs2 and TRAF6 expression (FIG. 4B). Thus, we validated our findings using splenic DCs pre-treated with NPs and activated with LPS. We found that NP_{ITE+Ins} increased SOCS2 expression, and concomitantly reduced TRAF6 levels in DCs (FIG. 4C). Moreover, although we did not detect an effect of NP_{ITE+Ins} on p38 MAPK or ERK1/2 activation, we detected reduced NF-κB p65 activation and translocation to the nucleus (FIG. 4D). To study the role of AhR in the induction of Socs2 expression by NP_{ITE+Ins} we used AhR-d DCs, which carry a hypomorphic AhR allele (Quintana et al., Nature 453, 65-71 (2008)). We found that deficient AhR signaling in AhR-d DCs abrogated the induction of Socs2 expression by NP_{ITE+Ins}. Taken together, these data suggest that NP_{ITE+Ins} modulates DC activation and function through the SOCS2-dependent inhibition of NF-κB signaling

NP_{ITE+Ins} Control DC Function Through the AhR-Dependent Induction of Socs2 Expression

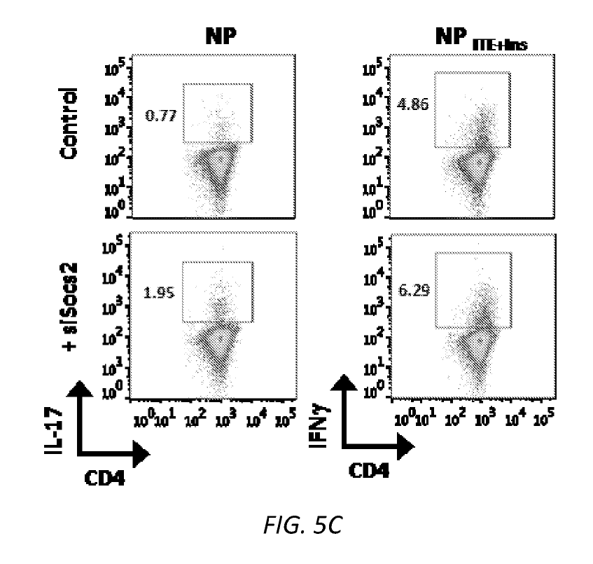

To further investigate the effects of socs2 expression on NF-κB signaling and DC function, we knocked down socs2 with small interfering RNAs (siRNAs) (FIG. 5A). The silencing of Socs2 in DCs was specific and did not alter the expression of Socs1 and Socs3, which can also inhibit the NF-κB activation. In agreement with its role in the regulation of TRAF6, the knock down of Socs2 increased TRAF6 expression as well as the activation and translocation of NF-κB p65 to the nucleus (FIG. 5B). Moreover, the knock down of socs2 abrogated the suppression of NF-κB p65 activation by NP_{ITE+Ins} and abrogated the ability of NP_{ITE+Ins} to suppress Th1 and Th17 cell differentiation induced by DCs (FIG. 5C). Taken together, these data suggest that the tolerogenic effects of NP_{ITE+Ins} involve the inhibition of NF-κB signaling through a SOCS2-dependent mechanism.

We then evaluated the effects of socs2 activity on DC activation and function. In agreement with the increased activation of NF-κB p65, the knock down of socs2 led to an increased expression of the co-stimulatory molecules CD40 and CD80 and MHC II, which are controlled by NF-κB p65. Moreover, the knock down of socs2 led to an augmented APC function, indicated by the increased activation BDC2.5 CD4+ T cells in the presence of MIMO. Altogether, these data demonstrate that the effects of NP_{ITE+Ins} in DCs are mediated by AhR-dependent induction of socs2.

NP_{ITE+GAD} Induce Tolerogenic DCs in Humans

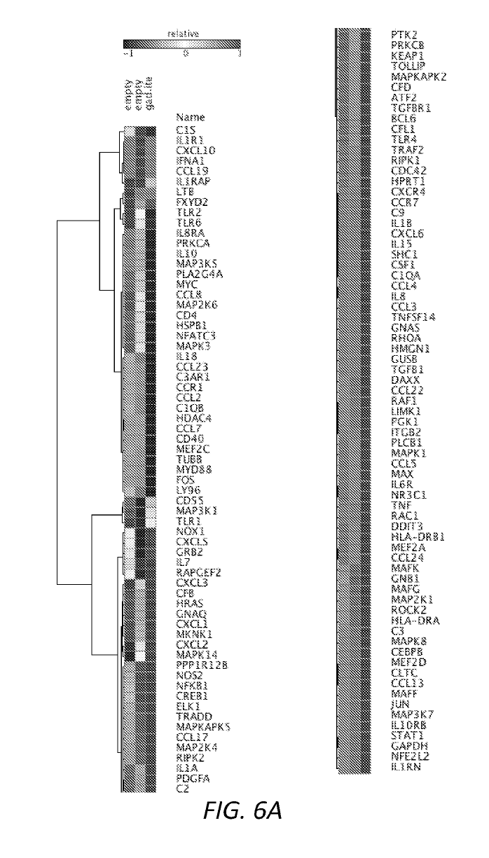

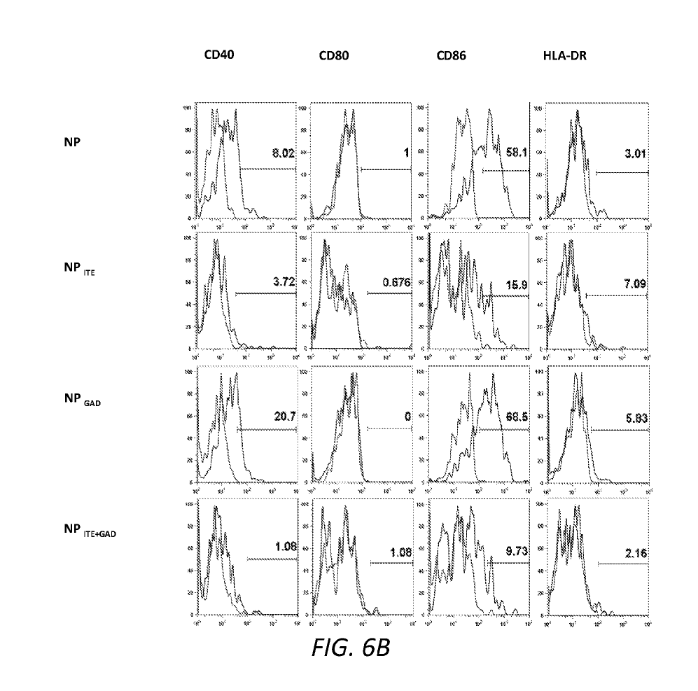

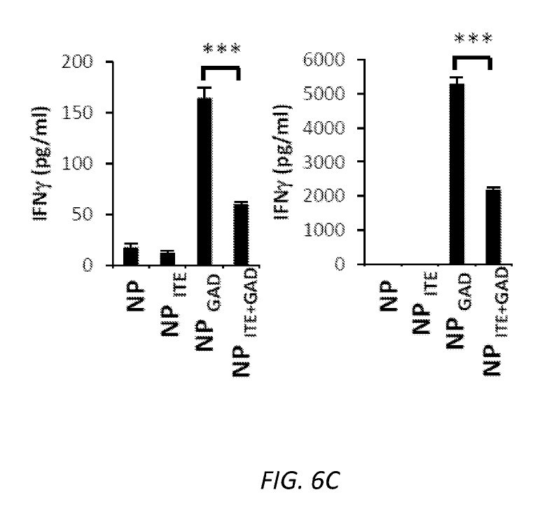

To evaluate the translational potential of NPs we studied their effects on human monocyte-derived dendritic cells (hDCs). For these studies we used NPs carrying ITE and glutamic acid decarboxylase (GAD₅₅₅₋₅₆₇), a major target in human T1D (NP_{ITE+GAD}). We found that NP_{ITE+GAD} activated AHR in hDCs, increasing the expression of AhR target gene CYP1A1, and treatment with NP_{ITE+GAD} modulated the expression of genes involved in APC activation and function (FIG. 6A). Indeed, treatment of DCs with NP_{ITE+GAD} decreased the expression of the costimulatory molecules CD40, CD86 and the antigen-presenting molecule HLA-DR (FIG. 6B), resembling our observations made on NOD DCs and suggesting that NP_{ITE+GAD} induce a tolerogenic phenotype in hDCs.

To investigate the functional relevance of these observations, we used NP-treated hDCs to activate a GAD-specific CD4+ T cell line isolated from a T1D subject. We found that treatment of mature or immature hDCs with NP_{ITE+GAD} decreased their ability to trigger IFNg production in T cells (FIG. 6C). Taken together, this data demonstrate that in vitro treatment with NP_{ITE+GAD} promotes the generation of tolerogenic hDCs with an impaired ability to trigger the activation of human diabetogenic T cells.

### REFERENCES

1. Atkinson M A, Eisenbarth G S, Michels A W. Type 1 diabetes. Lancet 2014, 383(9911): 69-82.
2. Bluestone J A, Herold K, Eisenbarth G. Genetics, pathogenesis and clinical interventions in type 1 diabetes. Nature 2010, 464(7293): 1293-1300.
3. Lehuen A, Diana J, Zaccone P, Cooke A. Immune cell crosstalk in type 1 diabetes. Nature reviews Immunology 2010, 10(7): 501-513.
4. Peakman M. Immunological pathways to beta-cell damage in Type 1 diabetes. Diabetic medicine: a journal of the British Diabetic Association 2013, 30(2): 147-154.
5. van Belle T L, Coppieters K T, von Herrath M G. Type 1 diabetes: etiology, immunology, and therapeutic strategies. Physiological reviews 2011, 91(1): 79-118.
6. Wallberg M, Cooke A. Immune mechanisms in type 1 diabetes. Trends in immunology 2013, 34(12): 583-591.
7. Bluestone J A, Thomson A W, Shevach E M, Weiner H L. What does the future hold for cell-based tolerogenic therapy? Nature reviews Immunology 2007, 7(8): 650-654.
8. Josefowicz S Z, Lu L F, Rudensky A Y. Regulatory T cells: mechanisms of differentiation and function. Annu Rev Immunol 2012, 30: 531-564.
9. Sakaguchi S, Miyara M, Costantino C M, Hafler D A. FOXP3+ regulatory T cells in the human immune system. Nature reviews Immunology 2010, 10(7): 490-500.
10. Ferraro A, Socci C, Stabilini A, Valle A, Monti P, Piemonti L, et al. Expansion of Th17 cells and functional defects in T regulatory cells are key features of the pancreatic lymph nodes in subjects with type 1 diabetes. Diabetes 2011, 60(11): 2903-2913.
11. Willcox A, Richardson S J, Bone A J, Foulis A K, Morgan N G. Analysis of islet inflammation in human type 1 diabetes. Clin Exp Immunol 2009, 155(2): 173-181.
12. Herold K C, Vignali D A, Cooke A, Bluestone J A. Type 1 diabetes: translating mechanistic observations into effective clinical outcomes. Nature reviews Immunology 2013, 13(4): 243-256.
13. Jeker L T, Bour-Jordan H, Bluestone J A. Breakdown in peripheral tolerance in type 1 diabetes in mice and humans. Cold Spring Harb Perspect Med 2012, 2(3): a007807.
14. Thompson J A, Perry D, Brusko T M. Autologous regulatory T cells for the treatment of type 1 diabetes. Curr Diab Rep 2012, 12(5): 623-632.
15. Battaglia M, Roncarolo M G. Immune intervention with T regulatory cells: past lessons and future perspectives for type 1 diabetes. Seminars in immunology 2011, 23(3): 182-194.
16. Coppieters K T, Harrison L C, von Herrath M G. Trials in type 1 diabetes: Antigen-specific therapies. Clinical immunology (Orlando, Fla.) 2013, 149(3): 345-355.
17. Creusot R J, Giannoukakis N, Trucco M, Clare-Salzler M J, Fathman C G. It's time to bring dendritic cell therapy to type 1 diabetes. Diabetes 2014, 63(1): 20-30.
18. Kim J H, Jin S M, Kim H S, Kim K A, Lee M S. Immunotherapeutic treatment of autoimmune diabetes. Critical reviews in immunology 2013, 33(3): 245-281.
19. Lee C N, Lew A M, Wu L. The potential role of dendritic cells in the therapy of Type 1 diabetes. Immunotherapy 2013, 5(6): 591-606.
20. Mukherjee G, Dilorenzo T P. The immunotherapeutic potential of dendritic cells in type 1 diabetes. Clin Exp Immunol 2010, 161(2): 197-207.
21. Lernmark A, Larsson H E. Immune therapy in type 1 diabetes mellitus. Nature reviews Endocrinology 2013, 9(2): 92-103.
22. Michels A W, Eisenbarth G S. Immune intervention in type 1 diabetes. Seminars in immunology 2011, 23(3): 214-219.
23. von Herrath M, Peakman M, Roep B. Progress in immune-based therapies for type 1 diabetes. Clin Exp Immunol 2013, 172(2): 186-202.
24. Apetoh L, Quintana F J, Pot C, Joller N, Xiao S, Kumar D, et al. The aryl hydrocarbon receptor interacts with c-Maf to promote the differentiation of type 1 regulatory T cells induced by IL-27. Nat Immunol 2010, 11(9): 854-861.
25. Gandhi R, Kumar D, Burns E J, Nadeau M, Dake B, Laroni A, et al. Activation of the aryl hydrocarbon receptor induces human type 1 regulatory T cell-like and Foxp3(+) regulatory T cells. Nat Immunol 2010, 11(9): 846-853.
26. Quintana F J, Basso A S, Iglesias A H, Korn T, Farez M F, Bettelli E, et al. Control of T(reg) and T(H)17 cell differentiation by the aryl hydrocarbon receptor. Nature 2008, 23: 23.
27. Quintana F J, Iglesias A H, Farez M F, Caccamo M, Burns E J, Kassam N, et al. Adaptive autoimmunity and Foxp3-based immunoregulation in zebrafish. PLoS One 2010, 5(3): e9478.
28. Quintana F J, Murugaiyan G, Farez M F, Mitsdoerffer M, Tukpah A M, Burns E J, et al. An endogenous aryl hydrocarbon receptor ligand acts on dendritic cells and T cells to suppress experimental autoimmune encephalomyelitis. Proc Natl Acad Sci USA 2010, 107(48): 20768-20773.
29. Quintana F J, Jin H, Burns E J, Nadeau M, Yeste A, Kumar D, et al. Aiolos promotes T(H)17 differentiation by directly silencing 112 expression. Nature Immunology 2012, 13(8): 770-777.
30. Yeste A, Nadeau M, Burns E J, Weiner H L, Quintana F J. Nanoparticle-mediated codelivery of myelin antigen and a tolerogenic small molecule suppresses experimental autoimmune encephalomyelitis. Proc Natl Acad Sci USA 2012, 109(28): 11270-11275.
31. Wu H Y, Quintana F J, da Cunha A P, Dake B T, Koeglsperger T, Starossom S C, et al. In Vivo Induction of Tr1 Cells via Mucosal Dendritic Cells and AHR Signaling. PLoS One 2011, 6(8): e23618.
32. Song J, Clagett-Dame M, Peterson R E, Hahn M E, Westler W M, Sicinski R R, et al. A ligand for the aryl hydrocarbon receptor isolated from lung. Proc Natl Acad Sci USA 2002, 99(23): 14694-14699 Epub 122 October 14630.
33. Benson J M, Shepherd D M. Dietary ligands of the aryl hydrocarbon receptor induce anti-inflammatory and immunoregulatory effects on murine dendritic cells. Toxicol Sci 2011, 124(2): 327-338.
34. Hauben E, Gregori S, Draghici E, Migliavacca B, Olivieri S, Woisetschlager M, et al. Activation of the aryl hydrocarbon receptor promotes allograft-specific tolerance through direct and dendritic cell-mediated effects on regulatory T cells. Blood 2008, 112(4): 1214-1222.
35. Mezrich J D, Fechner J H, Zhang X, Johnson B P, Burlingham W J, Bradfield C A. An interaction between kynurenine and the aryl hydrocarbon receptor can generate regulatory T cells. J Immunol 2010, 185(6): 3190-3198.
36. Nguyen N T, Kimura A, Nakahama T, Chinen I, Masuda K, Nohara K, et al. Aryl hydrocarbon receptor negatively regulates dendritic cell immunogenicity via a kynurenine-dependent mechanism. Proc Natl Acad Sci USA 2010, 107(46): 19961-19966.
37. Aryan P, Pietropaolo M, Ostrov D, Rhodes C J. Islet autoantigens: structure, function, localization, and regulation. Cold Spring Harb Perspect Med 2012, 2(8).
38. Fousteri G, Dave A, Bot A, Juntti T, Omid S, von Herrath M. Subcutaneous insulin B:9-23/IFA immunisation induces Tregs that control late-stage prediabetes in NOD mice through IL-10 and IFNgamma. Diabetologia 2010, 53(9): 1958-1970.
39. Roep B O, Peakman M. Antigen targets of type 1 diabetes autoimmunity. Cold Spring Harb Perspect Med 2012, 2(4): a007781.
40. Nakayama M, Abiru N, Moriyama H, Babaya N, Liu E, Miao D, et al. Prime role for an insulin epitope in the development of type 1 diabetes in NOD mice. Nature 2005, 435(7039): 220-223.
41. Katz J D, Wang B, Haskins K, Benoist C, Mathis D. Following a diabetogenic T cell from genesis through pathogenesis. Cell 1993, 74(6): 1089-1100.
42. Steinman R M. Decisions about dendritic cells: past, present, and future. Annu Rev Immunol 2012, 30: 1-22.
43. Mildner A, Jung S. Development and Function of Dendritic Cell Subsets. Immunity 2014, 40(5): 642-656.
44. Ganguly D, Haak S, Sisirak V, Reizis B. The role of dendritic cells in autoimmunity. Nature reviews Immunology 2013, 13(8): 566-577.
45. Mascanfroni I D, Yeste A, Vieira S M, Burns E J, Patel B, Sloma I, et al. IL-27 acts on DCs to suppress the T cell response and autoimmunity by inducing expression of the immunoregulatory molecule CD39. Nature immunology 2013, 14(10): 1054-1063.
46. Sosenko J M, Skyler J S, Palmer J P, Krischer J P, Yu L, Mahon J, et al. The prediction of type 1 diabetes by multiple autoantibody levels and their incorporation into an autoantibody risk score in relatives of type 1 diabetic subjects. Diabetes care 2013, 36(9): 2615-2620.
47. Quintana F J, Hagedorn P H, Elizur G, Merbl Y, Domany E, Cohen I R. Functional immunomics: microarray analysis of IgG autoantibody repertoires predicts the future response of mice to induced diabetes. Proc Natl Acad Sci USA 2004, 101 Suppl 2: 14615-14621.
48. Diana J, Simoni Y, Furio L, Beaudoin L, Agerberth B, Barrat F, et al. Crosstalk between neutrophils, B-1a cells and plasmacytoid dendritic cells initiates autoimmune diabetes. Nature medicine 2013, 19(1): 65-73.
49. McBerry C, Gonzalez R M, Shryock N, Dias A, Aliberti J. SOCS2-induced proteasome-dependent TRAF6 degradation: a common anti-inflammatory pathway for control of innate immune responses. PLoS One 2012, 7(6): e38384.
50. Boverhof D R, Tam E, Harney A S, Crawford R B, Kaminski N E, Zacharewski T R. 2,3,7,8-Tetrachlorodibenzo-p-dioxin induces suppressor of cytokine signaling 2 in murine B cells. Molecular pharmacology 2004, 66(6): 1662-1670.
51. Driver J P, Serreze D V, Chen Y G. Mouse models for the study of autoimmune type 1 diabetes: a NOD to similarities and differences to human disease. Seminars in immunopathology 2011, 33(1): 67-87.
52. Bending D, De la Pena H, Veldhoen M, Phillips J M, Uyttenhove C, Stockinger B, et al. Highly purified Th17 cells from BDC2.5NOD mice convert into Th1-like cells in NOD/SCID recipient mice. The Journal of clinical investigation 2009, 119(3): 565-572.
53. Esensten J H, Lee M R, Glimcher L H, Bluestone J A. T-bet-deficient NOD mice are protected from diabetes due to defects in both T cell and innate immune system function. J Immunol 2009, 183(1): 75-82.
54. Joseph J, Bittner S, Kaiser F M, Wiendl H, Kissler S. IL-17 silencing does not protect nonobese diabetic mice from autoimmune diabetes. J Immunol 2012, 188(1): 216-221.
55. D'Alise A M, Ergun A, Hill J A, Mathis D, Benoist C. A cluster of coregulated genes determines TGF-beta-induced regulatory T-cell (Treg) dysfunction in NOD mice. Proc Natl Acad Sci USA 2011, 108(21): 8737-8742.
56. Feuerer M, Shen Y, Littman D R, Benoist C, Mathis D. How punctual ablation of regulatory T cells unleashes an autoimmune lesion within the pancreatic islets. Immunity 2009, 31(4): 654-664.
57. Bresson D, Fousteri G, Manenkova Y, Croft M, von Herrath M. Antigen-specific prevention of type 1 diabetes in NOD mice is ameliorated by OX40 agonist treatment. Journal of autoimmunity 2011, 37(4): 342-351.
58. Grinberg-Bleyer Y, Baeyens A, You S, Elhage R, Fourcade G, Gregoire S, et al. IL-2 reverses established type 1 diabetes in NOD mice by a local effect on pancreatic regulatory T cells. The Journal of experimental medicine 2010, 207(9): 1871-1878.
59. Tsai S, Shameli A, Yamanouchi J, Clemente-Casares X, Wang J, Serra P, et al. Reversal of autoimmunity by boosting memory-like autoregulatory T cells. Immunity 2010, 32(4): 568-580.
60. Shameli A, Clemente-Casares X, Wang J, Santamaria P. Development of memory-like autoregulatory CD8+ T cells is CD4+ T cell dependent. J Immunol 2011, 187(6): 2859-2866.
61. Tsai S, Serra P, Clemente-Casares X, Slattery R M, Santamaria P. Dendritic cell-dependent in vivo generation of autoregulatory T cells by antidiabetogenic MHC class II. J Immunol 2013, 191(1): 70-82.
62. Ohkura N, Kitagawa Y, Sakaguchi S. Development and maintenance of regulatory T cells. Immunity 2013, 38(3): 414-423.
63. Roncarolo M G, Gregori S, Bacchetta R, Battaglia M. Tr1 cells and the counter-regulation of immunity: natural mechanisms and therapeutic applications. Current topics in microbiology and immunology 2014, 380: 39-68.
64. Quintana F J, Yeste A, Mascanfroni I D. Role and therapeutic value of dendritic cells in central nervous system autoimmunity. Cell death and differentiation 2014.
65. Bessede A, Gargaro M, Pallotta M T, Matino D, Servillo G, Brunacci C, et al. Aryl hydrocarbon receptor control of a disease tolerance defence pathway. Nature 2014, 511(7508): 184-190.
66. Nishio J, Feuerer M, Wong J, Mathis D, Benoist C. Anti-CD3 therapy permits regulatory T cells to surmount T cell receptor-specified peripheral niche constraints. The Journal of experimental medicine 2010, 207(9): 1879-1889.
67. Barrett J C, Clayton D G, Concannon P, Akolkar B, Cooper J D, Erlich H A, et al. Genome-wide association study and meta-analysis find that over 40 loci affect risk of type 1 diabetes. Nature genetics 2009, 41(6): 703-707.
68. Dissanayake D, Hall H, Berg-Brown N, Elford A R, Hamilton S R, Murakami K, et al. Nuclear factor-kappaB 1 controls the functional maturation of dendritic cells and prevents the activation of autoreactive T cells. Nature medicine 2011, 17(12): 1663-1667.
69. Huang G, Wang Y, Vogel P, Kanneganti T D, Otsu K, Chi H. Signaling via the kinase p38alpha programs dendritic cells to drive TH17 differentiation and autoimmune inflammation. Nat Immunol 2012, 13(2): 152-161.
70. Clemente-Casares X, Tsai S, Huang C, Santamaria P. Antigen-specific therapeutic approaches in Type 1 diabetes. Cold Spring Harb Perspect Med 2012, 2(2): a007773.
71. Quintana F J, Carmi P, Cohen I R. DNA vaccination with heat shock protein 60 inhibits cyclophosphamide-accelerated diabetes. J Immunol 2002, 169(10): 6030-6035.
72. Roep B O, Solvason N, Gottlieb P A, Abreu J R, Harrison L C, Eisenbarth G S, et al. Plasmid-encoded proinsulin preserves C-peptide while specifically reducing proinsulin-specific CD8(+) T cells in type 1 diabetes. Science translational medicine 2013, 5(191): 191ra182.
73. Solvason N, Lou Y P, Peters W, Evans E, Martinez J, Ramirez U, et al. Improved efficacy of a tolerizing DNA vaccine for reversal of hyperglycemia through enhancement of gene expression and localization to intracellular sites. J Immunol 2008, 181(12): 8298-8307.
74. Getts D R, Martin A J, McCarthy D P, Terry R L, Hunter Z N, Yap W T, et al. Microparticles bearing encephalitogenic peptides induce T-cell tolerance and ameliorate experimental autoimmune encephalomyelitis. Nat Biotechnol 2012, 30(12): 1217-1224.
75. Qian X, Peng X H, Ansari D O, Yin-Goen Q, Chen G Z, Shin D M, et al. In vivo tumor targeting and spectroscopic detection with surface-enhanced Raman nanoparticle tags. Nat Biotechnol 2008, 26(1): 83-90 Epub 27 Dec. 2023.
76. Gaglia J L, Guimaraes A R, Harisinghani M, Turvey S E, Jackson R, Benoist C, et al. Noninvasive imaging of pancreatic islet inflammation in type 1A diabetes subjects. The Journal of clinical investigation 2011, 121(1): 442-445.
77. Larman H B, Laserson U, Querol L, Verhaeghen K, Solimini N L, Xu G J, et al. PhIP-Seq characterization of autoantibodies from subjects with multiple sclerosis, type 1 diabetes and rheumatoid arthritis. Journal of autoimmunity 2013, 43: 1-9.
78. Mamchak A A, Manenkova Y, Leconet W, Zheng Y, Chan J R, Stokes C L, et al. Preexisting autoantibodies predict efficacy of oral insulin to cure autoimmune diabetes in combination with anti-CD3. Diabetes 2012, 61(6): 1490-1499.
79. Geiss G K, Bumgarner R E, Birditt B, Dahl T, Dowidar N, Dunaway D L, et al. Direct multiplexed measurement of gene expression with color-coded probe pairs. Nat Biotechnol 2008, 26(3): 317-325.
80. Quintana F J, Farez M F, Viglietta V, Iglesias A H, Merbl Y, Izquierdo G, et al. Antigen microarrays identify unique serum autoantibody signatures in clinical and pathologic subtypes of multiple sclerosis. Proc Natl Acad Sci USA 2008, 105(48): 18889-18894.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A composition comprising:
(i) one or more of:
a ligand that binds specifically to an aryl hydrocarbon receptor (AHR) transcription factor, an inhibitor of p38, or an inhibitor of Nuclear Factor kappa B (NF-kB); and
(ii) a diabetes autoantigen, wherein both (i) and (ii) are linked to a biocompatible nanoparticle.

2. The composition of claim 1, wherein the ligand that binds to AHR is a small molecule ligand of AHR.

3. The composition of claim 1, wherein the ligand that binds to AHR is 2-(1′H-indole-3′-carbonyl)-thiazole-4-carboxylic acid methyl ester (ITE).

4. The composition of claim 1, wherein the inhibitor of p38 is selected from the group consisting of SD282 (2-(6-chloro-5-((2R,5S)-4-(4-fluorobenzyl)-2,5-dimethylpiperazine-1-carbonyl)-1-methyl-1H-indol-3-yl)-N,N-dimethyl-2-oxoacetamide); 6-chloro-5-[[(2S,5R)-4-[(4-fluorophenyl)methyl]-2,5-domethyl-1-piperaziny-1]carbonyl]-N,N,1-trimethyl-.alpha.-oxo-1H-indole-3-acetamide; SKF86002 (6-(4-Fluorophenyl)-5-(4-pyridyl)-2,3-dihydroimidazo[2,1-b]-thiazole); PD169316 (4-[5-(4-fluorophenyl)-2-(4-nitrophenyl)-1H-imidazol-4-yl]-pyridine); SC68376 (2-Methyl-4-phenyl-5-(4-pyridyl)oxazole); VX702; VX745; R130823; AMG548; SCIO469; SCIO323; MW012069ASRM; SD169; RWJ67657; ARRY797; SB203580 (4-[4-(4-fluorophenyl)-2-(4-methylsulfinylphenyl)-1H-imidazol-5-yl]pyridine); LY 2228820 (5-(2-tert-butyl-4-(4-fluorophenyl)-1H-imidazol-5-yl)-3-neopentyl-3H-imidazo[4,5-b]pyridin-2-amine dimethanesulfonate); SB202190 (4-(4-fluorophenyl)-2-(4-hydroxyphenyl)-5-(4-pyridyl)-1H-imidazole) and derivatives thereof; SB239063 (trans-1-(4-Hydroxycyclohexyl)-4-(4-fluorophenyl)-5-(2-methoxypyridimidin-4-yl)imidazole); BMS 582949)4-[[5-[(cyclopropylamino)carbonyl]-2-methylphenyl]amino]-5-methyl-N-propylpyrrolo[2,1-f][1,2,4]triazine-6-carboxamide, see US20060235020); SB220025 and derivatives thereof; PD169316; RPR200765A; SB681323 (Dilmapimod); AMG548 (2-[[(2S)-2-amino-3-phenylpropyl]amino]-3-methyl-5-(2-naphthalenyl)-6-(4-pyridinyl)-4(3H)-pyrimidinone); ARRY-797; ARRY-371797; BIRB-796 (Doramapimod, 1-(3-tert-butyl-1-p-tolyl-1H-pyrazol-5-yl)-3-(4-(2-morpholinoethoxy)naphthalen-1-yl)urea); 856553 (Losmapimod, 6-[5-(cyclopropylcarbamoyl)-3-fluoro-2-methylphenyl]-N-(2,2-dimethylpropyl)pyridine-3-carboxamide); AZD6703; KC-706; PH 797804; R1503; SC-80036; SC1O-469; SC10-323; VX-702 or VX745 (5-(2,6-dichlorophenyl)-2-(phenylthio)-6H-pyrimido[1,6-b]pyridazin-6-one); and FR167653.

5. The composition of claim 1, wherein the inhibitor of NF-kB is selected from the group consisting of celastrol; dexamethasone; triptolide; CAY10512; helenalin; NFκB activation inhibitor II, JSH-23; andrographolide; sulfasalazine; rapamycin and rapamycin derivatives (e.g., temsirolimus and everolimus); caffeic acid phenethylester; SN50 (a cell-permeable inhibitory peptide); parthenolide; triptolide; wedelolactone; lactacystin; MG-132 [Z-Leu-Leu-Leu-H]. rocaglamide; sodium salicylate; pyrrolidinedithiocarbamic acid; substituted resorcinols, (E)-3-(4-methylphenylsulfonyl)-2-propenenitrile (Bay 11-7082); tetrahydrocurcuminoids (such as Tetrahydrocurcuminoid CG); lignans (manassantins, (+)-saucernetin, (−)-saucerneol methyl ether), sesquiterpenes (costunolide, parthenolide, celastrol, celaphanol A), diterpenes (excisanin, kamebakaurin), triterpenes (avicin, oleandrin), and polyphenols (resveratrol, epigallocatechin gallate, quercetin).

6. The composition of claim 1, wherein the diabetes autoantigen is selected from the group consisting of preproinsulin or an immunologically active fragment thereof, islet cell autoantigens (ICA), glutamic acid decarboxylase (GAD), IGRP, islet tyrosine phosphatase ICA512/IA-2, ICA12, ICA69, HSP60, HSP70, carboxypeptidase H, peripherin, and gangliosides, or immunologically active fragments thereof.

7. The composition of claim 1, wherein the diabetes autoantigen is selected from the group consisting of preproinsulin or an immunologically active fragment thereof, islet cell autoantigen (ICA), or GAD.

8. The composition of claim 1, further comprising a monoamine oxidase inhibitor (MAOI).

9. The composition of claim 1, further comprising an antibody that selectively binds to an antigen present on a T cell, a B cell, a dendritic cell, or a macrophage.

10. The composition of claim 9, wherein the antibody is linked to the biocompatible nanoparticle.

11. A method for increasing the number of CD4/CD25/Foxp3-expressing T regulatory (Treg) cells in a population of T cells, the method comprising:
contacting the population of cells with a sufficient amount of the composition of claim 1, and
optionally evaluating the presence and/or number of CD4/CD25/Foxp3-expressing cells in the population;
wherein the method results in an increase in the number and/or activity of regulatory T cells (Treg).

12. The method of claim 11, wherein the population of T cells comprises naïve T cells or CD4⁺CD62 ligand⁺ T cells.

13. The method of claim 11, further comprising administering the Treg cells to a subject suffering from or at risk of developing diabetes.

14. A method of treating, preventing, or reducing the risk of developing type 1 diabetes in a subject, the method comprising administering to the subject a therapeutically effective amount of the composition of claim 1.

15. The method of claim 14, comprising administering to the subject a therapeutically effective amount of the composition of claim 1, plus one or both of a monoamine oxidase inhibitor (MAOI), and an antibody that selectively binds to an antigen present on a T cell, a B cell, a dendritic cell, or a macrophage.

16. The method of claim 15, wherein the antibody is selected from the group consisting of antibodies that bind specifically to CXCR4, CD28, CD8, CTLA4, CD3, CD20, CD19, CD11c, DEC205, MHC class I or class II, CD80, CD86, CD11b, MHC class I or class II, CD80, or CD86.

17. The method of claim 15, wherein the MAOI is tranylcypromine.

18. The method of claim 14, wherein levels of IL-10 producing T cells (Tr1 cells) and/or IL-10 producing CD8 T cells are increased in the subject.

19.-23. (canceled)

24. The composition of claim 2, wherein the small molecule ligand of AHR is selected from the group consisting of 2,3,7,8 tetrachlorodibenzo-p-dioxin (TCDD), tryptamine (TA), 6 formylindolo[3,2 b]carbazole (FICZ), laquinimod, and/or 2-(1′H-indole-3′-carbonyl)-thiazole-4-carboxylic acid methyl ester (ITE).

25. The composition of claim 8, wherein the MAOI is tranylcypromine.


SEQUENCE LISTING (18 sequences)

SEQ ID NO: 1 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 2 - protein, 585 aa, Homo sapiens
MASPGSGFWSFGSEDGSGDSENPGTARAWCQVAQKFTGGIGNKLCALLYGDAEKPAESGGSQPPRAAARKAACACDQKPCSCSKVDVNYAFLHATDLLPACDGERPTLAFLQDVMNILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGGAISNMYAMMIARFKMFPEVKEKGMAALPRLIAFTSEHSHFSLKKGAAALGIGTDSVILIKCDERGKMIPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQNCNQMHASYLFQQDKHYDLSYDTGDKALQCGRHVDVFKLWLMWRAKGTTGFEAHVDKCLELAEYLYNIIKNREGYEMVFDGKPQHTNVCFWYIPPSLRTLEDNEERMSRLSKVAPVIKARMMEYGTTMVSYQPLGDKVNFFRMVISNPAATHQDIDFLIEEIERLGQDL

SEQ ID NO: 3 - protein, 979 aa, Homo sapiens
MRRPRRPGGLGGSGGLRLLLCLLLLSSRPGGCSAVSAHGCLFDRRLCSHLEVCIQDGLFGQCQVGVGQARPLLQVTSPVLQRLQGVLRQLMSQGLSWHDDLTQYVISQEMERIPRLRPPEPRPRDRSGLAPKRPGPAGELLLQDIPTGSAPAAQHRLPQPPVGKGGAGASSSLSPLQAELLPPLLEHLLLPPQPPHPSLSYEPALLQPYLFHQFGSRDGSRVSEGSPGMVSVGPLPKAEAPALFSRTASKGIFGDHPGHSYGDLPGPSPAQLFQDSGLLYLAQELPAPSRARVPRLPEQGSSSRAEDSPEGYEKEGLGDRGEKPASPAVQPDAALQRLAAVLAGYGVELRQLTPEQLSTLLTLLQLLPKGAGRNPGGVVNVGADIKKTMEGPVEGRDTAELPARTSPMPGHPTASPTSSEVQQVPSPVSSEPPKAARPPVTPVLLEKKSPLGQSQPTVAGQPSARPAAEEYGYIVTDQKPLSLAAGVKLLEILAEHVHMSSGSFINISVVGPALTFRIRHNEQNLSLADVTQQAGLVKSELEAQTGLQILQTGVGQREEAAAVLPQTAHSTSPMRSVLLTLVALAGVAGLLVALAVALCVRQHARQQDKERLAALGPEGAHGDTTFEYQDLCRQHMATKSLFNRAEGPPEPSRVSSVSSQFSDAAQASPSSHSSTPSWCEEPAQANMDISTGHMILAYMEDHLRNRDRLAKEWQALCAYQAEPNTCATAQGEGNIKKNRHPDFLPYDHARIKLKVESSPSRSDYINASPIIEHDPRMPAYIATQGPLSHTIADFWQMVWESGCTVIVMLTPLVEDGVKQCDRYWPDEGASLYHVYEVNLVSEHIWCEDFLVRSFYLKNVQTQETRTLTQFHFLSWPAEGTPASTRPLLDFRRKVNKCYRGRSCPIIVHCSDGAGRTGTYILIDMVLNRMAKGVKEIDIAATLEHVRDQRPGLVRSKDQFEFALTAVAEEVNAILKALPQ

SEQ ID NO: 4 - protein, 622 aa, Homo sapiens
MSMRSPISAQLALDGVGTMVNCTIKSEEKKEPCHEAPQGSATAAEPQPGDPARASQDSADPQAPAQGNFRGSWDCSSPEGNGSPEPKRPGASEAASGSQEKLDFNRNLKEVVPAIEKLLSSDWKERFLGRNSMEAKDVKGTQESLAEKELQLLVMIHQLSTLRDQLLTAHSEQKNMAAMLFEKQQQQMELARQQQEQIAKQQQQLIQQQHKINLLQQQIQQVNMPYVMIPAFPPSHQPLPVTPDSQLALPIQPIPCKPVEYPLQLLHSPPAPVVKRPGAMATHHPLQEPSQPLNLTAKPKAPELPNTSSSPSLKMSSCVPRPPSHGGPTRDLQSSPPSLPLGFLGEGDAVTKAIQDARQLLHSHSGALDGSPNTPFRKDLISLDSSPAKERLEDGCVHPLEEAMLSCDMDGSRHFPESRNSSHIKRPMNAFMVWAKDERRKILQAFPDMHNSSISKILGSRWKSMTNQEKQPYYEEQARLSRQHLEKYPDYKYKPRPKRTCIVEGKRLRVGEYKALMRTRRQDARQSYVIPPQAGQVQMSSSDVLYPRAAGMPLAQPLVEHYVPRSLDPNMPVIVNTCSLREEGEGTDDRHSVADGEMYRYSEDEDSEGEEKSDGELVVLTD

SEQ ID NO: 5 - protein, 483 aa, Homo sapiens [sequence omitted]

SEQ ID NO: 6 - protein, 471 aa, Homo sapiens
MSHHPSGLRAGFSSTSYRRTFGPPPSLSPGAFSYSSSSRFSSSRLLGSASPSSSVRLGSFRSPRAGAGALLRLPSERLDFSMAEALNQEFLATRSNEKQELQELNDRFANFIEKVRFLEQQNAALRGELSQARGQEPARADQLCQQELRELRRELELLGRERDRVQVERDGLAEDLAALKQRLEEETRKREDAEHNLVLFRKDVDDATLSRLELERKIESLMDEIEFLKKLHEEELRDLQVSVESQQVQQVEVEATVKPELTAALRDIRAQYESIAAKNLQEAEEWYKSKYADLSDAANRNHEALRQAKQEMNESRRQIQSLTCEVDGLRGTNEALLRQLRELEEQFALEAGGYQAGAARLEEELRQLKEEMARHLREYQELLNVKMALDIEIATYRKLLEGEESRISVPVHSFASLNIKTTVPEVEPPQDSHSRKTVLIKTIETRNGEQVVTESQKEQRSELDKSSAHSY

SEQ ID NO: 7 - protein, 355 aa, Homo sapiens
MDFLHRNGVLIIQHLQKDYRAYYTFLNFMSNVGDPRNIFFIYFPLCFQFNQTVGTKMIWVAVIGDWLNLIFKWILFGHRPYWWVQETQIYPNHSSPCLEQFPTTCETGPGSPSGHAMGASCVWYVMVTAALSHTVCGMDKFSITLHRLTWSFLWSVFWLIQISVCISRVFIATHFPHQVILGVIGGMLVAEAFEHTPGIQTASLGTYLKTNLFLFLFAVGFYLLLRVLNIDLLWSVPIAKKWCANPDWIHIDTTPFAGLVRNLGVLFGLGFAINSEMFLLSCRGGNNYTLSFRLLCALTSLTILQLYHFLQIPTHEEHLFYVLSFCKSASIPLTVVAFIPYSVHMLMKQSGKKSQ

SEQ ID NO: 8 - protein, 154 aa, Homo sapiens
MDFLHRNGVLIIQHLQKDYRAYYTFLNFMSNVGDPRNIFFIYFPLCFQFNQTVGTKMIWVAVIGDWLNLIFKWILFGHRPYWWVQETQIYPNHSSPCLEQFPTTCETGPGSPSGHAMGASCVWYVMVTAALSHTVCGMDKFSITLHRHAGGRGL

SEQ ID NO: 9 - protein, 9 aa, Artificial Sequence
  Fragment of IGRP
VLFGLGFAI

SEQ ID NO: 10 - protein, 9 aa, Artificial Sequence
  Fragment of IGRP
NLFLFLFAV

SEQ ID NO: 11 - protein, 9 aa, Artificial Sequence
  Fragment of IGRP
FLFAVGFYL

SEQ ID NO: 12 - protein, 9 aa, Artificial Sequence
  Fragment of IGRP
YLLLRVLNI

SEQ ID NO: 13 - DNA, 18 bp, Artificial Sequence
  synthetically generated primer
ggaatggagcggacagga

SEQ ID NO: 14 - DNA, 18 bp, Artificial Sequence
  synthetically generated primer
ggaatggagcggacagga

SEQ ID NO: 15 - DNA, 20 bp, Artificial Sequence
  synthetically generated primer
atgagtcaacacgtcccaga

SEQ ID NO: 16 - DNA, 20 bp, Artificial Sequence
  synthetically generated primer
ctgcacactctcgttttggg

SEQ ID NO: 17 - DNA, 19 bp, Artificial Sequence
  synthetically generated primer
tggcaaagtctctcgcaga

SEQ ID NO: 18 - DNA, 20 bp, Artificial Sequence
  synthetically generated primer
tgctcggggttaaatggtac